(19) United States
(12) Patent Application Publication
(10) Pub. No.: US 2014/0162974 A1
(43) Pub. Date: Jun. 12, 2014

(54) METHODS FOR MODULATION OF PHOSPHORUS AND FGF23

(71) Applicant: Luitpold Pharmaceuticals, Inc., Shirley, NY (US)

(72) Inventors: David B Bregman, Clarksburg, NJ (US); Marc L Tokars, Douglassville, PA (US)

(73) Assignee: LUITPOLD PHARMACEUTICALS, INC., Shirley, NY (US)

(21) Appl. No.: 13/821,072

(22) PCT Filed: Mar. 5, 2013
(86) PCT No.: PCT/US13/29136

§ 371 Date: Mar. 6, 2013

Related U.S. Application Data
(60) Provisional application No. 61/606,645, filed on Mar. 5, 2012.
(60) Provisional application No. 61/768,046, filed on Feb. 22, 2013.

(51) Int. Cl. A61K 31/721; A61K 31/7135

(52) U.S. Cl. CPC A61K 31/721; A61K 31/7135
USPC 514/53; 514/59

(57) ABSTRACT

Compositions and methods for decreasing phosphorus, decreasing cFGF23, or increasing iFGF23 in a subject. Such compositions and methods can be useful for treatment of conditions, disorders, or diseases associated with phosphorus or FGF23 disregulation.

### CROSS-REFERENCE TO RELATED APPLICATIONS

This application claims priority from U.S. Provisional Application Ser. No. 61/606,645, filed on 5 Mar. 2012, and U.S. Provisional Application Ser. No. 61/768,046, filed on 22 Feb. 2013, each of which is incorporated herein by reference in its entirety.

### FIELD OF THE INVENTION

The present disclosure generally relates to reduction of phosphorus, reduction of C-terminal FGF23 (cFGF23), or increase of intact FGF23 (iFGF23) in a subject via administration of an iron carbohydrate complex.

### BACKGROUND OF THE INVENTION

Chronic kidney disease (CKD), also known as chronic renal disease, is a progressive loss in renal function over a period of months or years. CKD is associated with reduced phosphate excretion, leading to hyperphosphatemia, an electrolyte disturbance in which there is an abnormally elevated level of phosphate in the blood. High phosphate levels are conventionally treated with phosphate binders or dietary restriction of phosphate.

Chronic kidney disease can be associated with increased cardiovascular risk (Faul et al. 2011 J Clin Investig 121(11), 4393-4408). It has been suggested that higher levels of phosphorus in the blood are linked to increased calcification of the coronary arteries, a key marker of heart disease risk. It has also been reported that elevated FGF-23 is linked to greater risk of left ventricular hypertrophy in CKD patients (Faul et al. 2011 J Clin Investig 121(11), 4393-4408). It has also been reported that elevated FGF23 levels increase fractional phosphate excretion and reduce serum phosphate levels (Juppner 2011 Kidney Intl 79(Suppl 121), S24-S27).

Parenteral iron therapy is known to be effective in a variety of diseases and conditions including, but not limited to, severe iron deficiency, iron deficiency anemia, problems of intestinal iron absorption, oral iron intolerance, cases where patient compliance with an oral iron regimen is not guaranteed, iron deficiency where there is no response to oral therapy (e.g., dialysis patients), and situations where iron stores are scarcely or not at all formed but would be important for further therapy (e.g., in combination with erythropoietin) (Geisser et al. 1992 Arzneimittelforschung 42(12), 1439-1452). There exist various commercially available parenteral iron formulations. Currently available parenteral iron formulations approved for use in the U.S. include iron dextran (e.g., InFed, Dexferrum), sodium ferric gluconate complex in sucrose (Ferrlecit), and iron sucrose (Venofer). Treatment with an iron carbohydrate complex of patients having chronic heart failure and iron deficiency has been reported (Anker et al. 2009 N Engl J Med 361(25), 2436-2448).

### SUMMARY OF THE INVENTION

Among the various aspects of the present disclosure is the provision of a method for reduction of phosphorus, reduction of C-terminal FGF23 (cFGF23), or increase of intact FGF23 (iFGF23) in a subject. Reduction of phosphorus, reduction of C-terminal FGF23 (cFGF23), or increase of intact FGF23 (iFGF23) in a subject can be efficacious for the treatment of associated conditions, such as chronic kidney disease, hypophosphotemia, or heart disease or conditions.

One aspect provides a method of treating a disease or disorder associated with increased phosphorus or increased cFGF23 levels in a subject.

Another aspect provides a method of modulating phosphorus or FGF23 in a subject.

In some embodiments, the method includes administering an iron carbohydrate complex to a subject in need thereof; wherein, a phosphorus level of the subject decreases; a fractional excretion of phosphate of the subject increases; a C-terminal FGF23 (cFGF23) level of the subject decreases; or an intact FGF23 (iFGF23) level of the subject increases.

In some embodiments, the iron carbohydrate complex includes maltose; the phosphorus level of the subject decreases; the fractional excretion of phosphate of the subject increases; the C-terminal FGF23 (cFGF23) level of the subject decreases; and the intact FGF23 (iFGF23) level of the subject increases.

In some embodiments, the disease or disorder includes chronic kidney disease, hypophosphotemia, or a cardiac disease or condition. In some embodiments, the subject is diagnosed with an elevated level of phosphorus. In some embodiments, the subject is diagnosed with an elevated level of cFGF23. In some embodiments, the subject is diagnosed with an elevated level of phosphorus and an elevated level of cFGF23. In some embodiments, the subject is diagnosed with chronic kidney disease, hypophosphotemia, or a cardiac disease or condition. In some embodiments, the subject is diagnosed with a phosphorus level above a baseline. In some embodiments, the subject is diagnosed with a cFGF23 level above a baseline.

In some embodiments, the cardiac disease or condition is selected from the group consisting of: chronic heart damage, chronic heart failure, cardiac damage resulting from injury or trauma, cardiac damage resulting from a cardiotoxin, cardiac damage from radiation or oxidative free radicals, cardiac damage resulting from decreased blood flow, and myocardial infarction. In some embodiments, the cardiotoxin comprises cFGF23.

In some embodiments, the phosphorus level of the subject decreases by at least about 10%, at least about 20%, at least about 30%, at least about 40%, at least about 50%, at least about 60%, at least about 70%, at least about 80%, at least about 90%, or at least about 100%. In some embodiments, the phosphorus level of the subject decreases by at least about 0.01 mg/dL, at least about 0.05 mg/dL, at least about 0.1 mg/dL, at least about 0.2 mg/dL, at least about 0.3 mg/dL, at least about 0.4 mg/dL, at least about 0.5 mg/dL, at least about 0.6 mg/dL, at least about 0.7 mg/dL, at least about 0.8 mg/dL, at least about 0.9 mg/dL, at least about 1.0 mg/dL, at least about 1.1 mg/dL, at least about 1.2 mg/dL, at least about 1.3 mg/dL, at least about 1.4 mg/dL, at least about 1.5 mg/dL, at least about 1.6 mg/dL, at least about 1.7 mg/dL, at least about 1.8 mg/dL, at least about 1.9 mg/dL, at least about 2.0 mg/dL, at least about 2.1 mg/dL, at least about 2.2 mg/dL, at least about 2.3 mg/dL, at least about 2.4 mg/dL, at least about 2.5 mg/dL, at least about 2.6 mg/dL, at least about 2.7 mg/dL, at least about 2.8 mg/dL, at least about 2.9 mg/dL, at least about 3.0 mg/dL, at least about 3.1 mg/dL, at least about 3.2 mg/dL, at least about 3.3 mg/dL, at least about 3.4 mg/dL, or at least about 3.5 mg/dL.

In some embodiments, the fractional excretion of phosphate increases by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 40%, at least about 50%, at least about 60%, at least about 70%, at least about 80%, at least about 90%, or at least about 100%.

In some embodiments, the cFGF23 level of the subject decreases by at least about 10%, at least about 20%, at least about 30%, at least about 40%, at least about 50%, at least about 60%, at least about 70%, at least about 80%, at least about 90%, or at least about 100%. In some embodiments, the cFGF23 level of the subject decreases by at least about 50 RU/mL, at least about 100 RU/mL, at least about 150 RU/mL, at least about 200 RU/mL, at least about 250 RU/mL, at least about 300 RU/mL, at least about 350 RU/mL, at least about 400 RU/mL, at least about 450 RU/mL, at least about 500 RU/mL, at least about 550 RU/mL, at least about 600 RU/mL, at least about 650 RU/mL, at least about 700 RU/mL, at least about 750 RU/mL, at least about 800 RU/mL, at least about 850 RU/mL, at least about 900 RU/mL, at least about 1,000 RU/mL; at least about 1,100 RU/mL, at least about 1,200 RU/mL, at least about 1,300 RU/mL, at least about 1,400 RU/mL, at least about 1,500 RU/mL, at least about 1,600 RU/mL, at least about 1,700 RU/mL, at least about 1,800 RU/mL, at least about 1,900 RU/mL, or at least about 2,000 RU/mL.

In some embodiments, the iFGF23 level of the subject increases by at least about 10%, at least about 20%, at least about 30%, at least about 40%, at least about 50%, at least about 60%, at least about 70%, at least about 80%, at least about 90%, or at least about 100%. In some embodiments, the iFGF23 level of the subject increases by at least about 10 RU/mL, at least about 20 RU/mL, at least about 30 RU/mL, at least about 40 RU/mL, at least about 50 RU/mL, at least about 60 RU/mL, at least about 70 RU/mL, at least about 80 RU/mL, at least about 90 RU/mL, at least about 100 RU/mL, at least about 110 RU/mL, at least about 120 RU/mL, at least about 130 RU/mL, at least about 140 RU/mL, at least about 150 RU/mL, at least about 200 RU/mL, at least about 250 RU/mL, at least about 300 RU/mL, at least about 350 RU/mL, at least about 400 RU/mL, at least about 450 RU/mL, at least about 500 RU/mL, at least about 550 RU/mL, at least about 600 RU/mL, at least about 650 RU/mL, at least about 700 RU/mL, at least about 750 RU/mL, at least about 800 RU/mL, at least about 850 RU/mL, at least about 900 RU/mL, or at least about 1,000 RU/mL.

In some embodiments, the iFGF23 level of the subject increases by at least about 10 pg/mL, at least about 20 pg/mL, at least about 30 pg/mL, at least about 40 pg/mL, at least about 50 pg/mL, at least about 60 pg/mL, at least about 70 pg/mL, at least about 80 pg/mL, at least about 90 pg/mL, at least about 100 pg/mL, at least about 110 pg/mL, at least about 120 pg/mL, at least about 130 pg/mL, at least about 140 pg/mL, at least about 150 pg/mL, at least about 160 pg/mL, at least about 170 pg/mL, at least about 180 pg/mL, at least about 190 pg/mL, at least about 200 pg/mL, at least about 250 pg/mL, at least about 300 pg/mL, at least about 350 pg/mL, at least about 400 pg/mL, at least about 450 pg/mL, at least about 500 pg/mL, at least about 550 pg/mL, at least about 600 pg/mL, at least about 650 pg/mL, at least about 700 pg/mL, at least about 750 pg/mL, at least about 800 pg/mL, at least about 850 pg/mL, at least about 900 pg/mL, or at least about 1,000 pg/mL.

In some embodiments, the iron carbohydrate complex comprises one of: an iron carboxymaltose or an iron dextran. In some embodiments, the iron carbohydrate complex comprises maltose.

In some embodiments, the iron carbohydrate complex comprises an iron carboxymaltose; the phosphorus level of the subject decreases by at least about 0.01 mg/dL, at least about 0.05 mg/dL, at least about 0.1 mg/dL, at least about 0.2 mg/dL, at least about 0.3 mg/dL, at least about 0.4 mg/dL, at least about 0.5 mg/dL, at least about 0.6 mg/dL, at least about 0.7 mg/dL, at least about 0.8 mg/dL, at least about 0.9 mg/dL, at least about 1.0 mg/dL, at least about 1.1 mg/dL, at least about 1.2 mg/dL, at least about 1.3 mg/dL, at least about 1.4 mg/dL, or at least about 1.5 mg/dL; the cFGF23 level of the subject decreases by at least about 100 RU/mL, at least about 150 RU/mL, at least about 200 RU/mL, at least about 250 RU/mL, at least about 300 RU/mL, at least about 350 RU/mL, at least about 400 RU/mL, at least about 450 RU/mL, at least about 500 RU/mL, at least about 550 RU/mL, at least about 600 RU/mL, at least about 650 RU/mL, at least about 700 RU/mL, at least about 750 RU/mL, at least about 800 RU/mL, at least about 850 RU/mL, at least about 900 RU/mL, or at least about 1,000 RU/mL; and the iFGF23 level of the subject increases by at least about 10 pg/mL, at least about 20 pg/mL, at least about 30 pg/mL, at least about 40 pg/mL, at least about 50 pg/mL, at least about 60 pg/mL, at least about 70 pg/mL, at least about 80 pg/mL, at least about 90 pg/mL, at least about 100 pg/mL, at least about 110 pg/mL, at least about 120 pg/mL, at least about 130 pg/mL, at least about 140 pg/mL, at least about 150 pg/mL, at least about 160 pg/mL, at least about 170 pg/mL, at least about 180 pg/mL, at least about 190 pg/mL, at least about 200 pg/mL, at least about 250 pg/mL, at least about 300 pg/mL, at least about 350 pg/mL, at least about 400 pg/mL, at least about 450 pg/mL, at least about 500 pg/mL, at least about 550 pg/mL, at least about 600 pg/mL, at least about 650 pg/mL, at least about 700 pg/mL, at least about 750 pg/mL, at least about 800 pg/mL, at least about 850 pg/mL, at least about 900 pg/mL, or at least about 1,000 pg/mL.

In some embodiments, the iron carbohydrate complex comprises an iron dextran; the phosphorus level of the subject decreases by at least about 0.001 mg/dL, at least about at least about 0.005 mg/dL, at least about 0.01 mg/dL, at least about 0.15 mg/dL, or at least about 0.2 mg/dL; and the cFGF23 level of the subject decreases by at least about 100 RU/mL, at least about 150 RU/mL, at least about 200 RU/mL, at least about 250 RU/mL, at least about 300 RU/mL, at least about 350 RU/mL, at least about 400 RU/mL, at least about 450 RU/mL, at least about 500 RU/mL, at least about 550 RU/mL, at least about 600 RU/mL, at least about 650 RU/mL, at least about 700 RU/mL, at least about 750 RU/mL, at least about 800 RU/mL, at least about 850 RU/mL, at least about 900 RU/mL, or at least about 1,000 RU/mL.

In some embodiments, the subject is a mammalian subject. In some embodiments, the subject is a horse, cow, dog, cat, sheep, pig, mouse, rat, monkey or other primate, guinea pig, chicken, or human. In some embodiments, the subject is a human subject.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a line and scatter plot showing mean changes in phosphate (left vertical axis) or changes in FGF-23 (right vertical axis) with respect to baseline value at each study day (0, 7, 14, 28, or 56) as indicated on the Horizontal (X) axis for trial VIT30. FIG. 1 shows that the changes for phosphate for both the FCM and Venofer were decreased with respect to baseline values, which get more negative (larger decreases) on Day 14 with respect to Day 7 and then get less negative at each subsequent Visit. The magnitude of the changes for FGF-23 were almost always decreases with respect to baseline (except for FCM on Day 14). Further details regarding methodology are provided in Example 2.

FIG. 2A is a schematic of the study design. Further details are provided in Example 3.

FIG. 2B is a CONSORT diagram of flow of subjects. The safety population included all subjects who received study drug regardless of subsequent laboratory testing. The evaluable population included subjects with baseline and at least one post-randomization set of FGF23 and blood and urinary phosphate levels. Further details are provided in Example 3.

FIG. 3A is a line and scatter plot showing the effect of FCM and iron dextran on hemoglobin. Further details regarding methodology are provided in Example 3.

FIG. 3B is a line and scatter plot showing the effect of FCM and iron dextran on serum ferritin; *connotes p<0.05 for between group differences in change from baseline values. Further details regarding methodology are provided in Example 3.

FIG. 4A is a line and scatter plot showing the effect of FCM and iron dextran on serum phosphate; *connotes p<0.05 for between group differences in change from baseline values. Further details regarding methodology are provided in Example 3.

FIG. 4B is a line and scatter plot showing the effect of FCM and iron dextran on urinary fractional excretion of phosphate; *connotes p<0.05 for between group differences in change from baseline values. Further details regarding methodology are provided in Example 3.

FIG. 4C is a line and scatter plot showing the effect of FCM and iron dextran on plasma C-terminal FGF23. Further details regarding methodology are provided in Example 3.

FIG. 4D is a line and scatter plot showing the effect of FCM and iron dextran on serum intact FGF23; *connotes p<0.05 for between group differences in change from baseline values. Further details regarding methodology are provided in Example 3.

FIG. 5A is a line and scatter plot showing the effect of FCM and iron dextran on serum 25-hydroxyvitamin D. Further details regarding methodology are provided in Example 3.

FIG. 5B is a line and scatter plot showing the effect of FCM and iron dextran on serum 1,25-dihydroxyvitamin D; *connotes p<0.05 for between group differences in change from baseline values. Further details regarding methodology are provided in Example 3.

FIG. 5C is a line and scatter plot showing the effect of FCM and iron dextran on serum calcium; *connotes p<0.05 for between group differences in change from baseline values. Further details regarding methodology are provided in Example 3.

FIG. 5D is a line and scatter plot showing the effect of FCM and iron dextran on plasma PTH. Further details regarding methodology are provided in Example 3.

FIG. 6A is a line and scatter plot showing the effect of FCM on serum phosphate among subjects who did or did not develop serum phosphate<2.0 mg/dL; *connotes p<0.05 for between group differences in change from baseline values. Further details regarding methodology are provided in Example 3.

FIG. 6B is a line and scatter plot showing the effect of FCM on plasma C-terminal FGF23 among subjects who did or did not develop serum phosphate<2.0 mg/dL. Further details regarding methodology are provided in Example 3.

FIG. 6C is a line and scatter plot showing the effect of FCM on serum intact FGF23 among subjects who did or did not develop serum phosphate<2.0 mg/dL; *connotes p<0.05 for between group differences in change from baseline values. Further details regarding methodology are provided in Example 3.

FIG. 6D is a line and scatter plot showing the effect of FCM on urinary fractional excretion of phosphate among subjects who did or did not develop serum phosphate<2.0 mg/dL; *connotes p<0.05 for between group differences in change from baseline values. Further details regarding methodology are provided in Example 3.

FIG. 7A is a line and scatter plot showing the effect of FCM on serum 25-dihydroxyvitamin D among subjects who did or did not develop serum phosphate<2.0 mg/dL. Further details regarding methodology are provided in Example 3.

FIG. 7B is a line and scatter plot showing the effect of FCM on serum 1,25-dihydroxyvitamin D among subjects who did or did not develop serum phosphate<2.0 mg/dL; *connotes p<0.05 for between group differences in change from baseline values. Further details regarding methodology are provided in Example 3.

FIG. 7C is a line and scatter plot showing the effect of FCM on serum calcium among subjects who did or did not develop serum phosphate<2.0 mg/dL; *connotes p<0.05 for between group differences in change from baseline values. Further details regarding methodology are provided in Example 3.

FIG. 7D is a line and scatter plot showing the effect of FCM on plasma PTH among subjects who did or did not develop serum phosphate<2.0 mg/dL; *connotes p<0.05 for between group differences in change from baseline values. Further details regarding methodology are provided in Example 3.

FIG. 8A is an illustration showing fgf23 transcription in osteocytes is up-regulated by iron deficiency, but a counterbalancing increase in posttranslational FGF23 cleavage maintains normal net production of intact protein. Increased fgf23 transcription accompanied by increased intracellular FGF23 cleavage results in markedly elevated levels of FGF23 fragments that are detectable by the C-terminal assay.

FIG. 8B is an illustration showing correction of iron deficiency with iron dextran restores normal fgf23 transcription thereby decreasing production of FGF23 fragments while maintaining normal production of intact protein.

FIG. 8C is an illustration showing correction of iron deficiency with ferric carboxymaltose restores normal fgf23 transcription, but production of intact FGF23 protein increases nevertheless, perhaps because of a greater magnitude of concomitant inhibition of FGF23 cleavage by carboxymaltose.

FIG. 9 is a diagram illustrating the proposed mechanism for differential effects of iron deficiency and its correction with ferric carboxymaltose (FCM) or iron dextran on regulation of intact FGF23 (iFGF23) and C-terminal FGF23 (cFGF23) levels.

Further details regarding the proposed mechanism are provided in Example 3.

### DETAILED DESCRIPTION OF THE PREFERRED EMBODIMENTS

The present disclosure is based on, at least in part, the discovery that certain iron carbohydrate complexes can simultaneously decrease levels of phosphorus, decrease levels of C-terminal FGF23 (cFGF23), or increase levels of intact FGF23 (iFGF23) in a subject. Furthermore, it has been discovered that different classes of iron carbohydrate complexes can have a differential effect on phosphorus decrease, cFGF23 decrease, or iFGF23 increase.

Specifically, experiments reported herein with iron deficient women treated with ferric carboxymaltose (FCM) or iron dextran showed that: iron deficiency was associated with markedly elevated C-terminal FGF23 (cFGF23) but normal intact-FGF23 (iFGF23) levels; both FCM and iron dextran intravenous iron rapidly lowered cFGF23 levels by 80% within 24 hours; and FCM but not iron dextran induced a significant increase in iFGF23, which was associated with increased urinary fractional excretion of phosphate and decreased serum phosphate (see Examples). These data are the first direct evidence that iron deficiency can cause elevated cFGF23 in humans and can help explain the seemingly paradoxical findings that iron deficiency induces fgf23 transcription, whereas certain forms of high-dose iron therapy raise circulating FGF23 levels despite correcting iron deficiency.

While under no obligation to provide a mechanism, and in no way limiting the scope of the invention or the present disclosure, it is thought that intravenous iron lowers cFGF23 in humans by reducing fgf23 transcription (as in mice), whereas carbohydrate moieties in certain iron preparations may simultaneously inhibit FGF23 degradation in osteocytes leading to transient increases in iFGF23 and reduced serum phosphate. While under no obligation to provide a mechanism, and in no way limiting the scope of the invention or the present disclosure, it is thought that reduced serum phosphate observed after administration of some types of intravenous iron is driven by induction of iFGF23. For example, iFGF23 may block reabsorption of phosphate in kidney tubules and urinary fractional excretion of phosphate.

Until the present disclosure, one of ordinary skill in the would have avoided using a ferric carboxymaltose to lower phosphate levels or treat hypophosphotemia (or other phosphate-level associated diseases or disorders) at least because of a perceived risk of kidney damage. But as shown herein, ferric carboxymaltose reduces phosphate levels by inducing iFGF23 which leads to reabsorption of phosphate in kidney tubules and urinary fractional excretion of phosphate. In other words, counter to the understanding in the art, the present disclosure establishes that ferric carboxymaltose can lower phosphate levels or treat hypophosphotemia safely and without kidney damage.

Provided herein is a therapeutic method for simultaneously reducing phosphorus, reducing cFGF23, or increasing iFGF23 in a subject by administering an iron carbohydrate complex. For example, administering a maltose-containing iron carbohydrate, such as ferric carboxymaltose, to a subject can reduce phosphorus, reduce cFGF23, and increase iFGF23. As another example, administering an iron dextran to a subject can reduce cFGF23. Generally, states indicative of a need for reduction in phosphorus, reduction in cFGF23, or increase in iFGF23 levels include, but are not limited to, chronic kidney disease or damaged or degenerated heart tissue. For example, elevated levels of cFGF23 is associated with poor outcome in hemodialysis and non-hemodialysis dependent chronic kidney disease (see e.g., Gutierrez et al. 2008 N Engl J Med 359:584-592); and such subjects can benefit from reduction in cFGF23 levels according to protocols described herein. As another example, a subject having chronic kidney disease may have too high of levels of serum phosphate for conventional phosphate binders to be effective; and such subjects can benefit from reduction in phosphate levels according to protocols described herein. As another example, a subject having chronic kidney disease and hypophosphotemia can benefit from reduction in phosphate levels according to protocols described herein.

Furthermore, the level of phosphorus reduction can be modulated through choice of iron carbohydrate complex. For example, an iron carboxymaltose can more significantly reduce phosphorus as compared to an iron dextran or an iron sucrose. As another example, an iron dextran can more significantly reduce phosphorus as compared to an iron sucrose. Such differential modulation of phosphorus decrease can be useful according to a subject's diagnosed phosphorus level.

Furthermore, the level of FGF23 reduction can be modulated through choice of iron carbohydrate complex. For example, an iron carboxymaltose or an iron dextran can more significantly reduce FGF23 as compared to an iron sucrose. Such differential modulation of FGF23 decrease can be useful according to a subject's diagnosed FGF23 level.

Phosphorus

As described herein, administration of an iron carbohydrate complex can decrease phosphorus levels in a subject. For example, administration of a maltose-containing iron carbohydrate complex can decrease phosphorus levels in a subject.

Processes for determining a phosphorus level of a subject or of a biological sample are well known (see e.g., Examples 1-2). For example, serum phosphorus assays are part of standard routine chemistry evaluations. Except as otherwise noted herein, therefore, the process of the present disclosure can be carried out in accordance with such processes.

As described herein, an iron carbohydrate complex can reduce elevated phosphorus in a subject. An elevated level of phosphorus in a subject can be in comparison to a reference or baseline value for phosphorus. A reference or baseline value can be an average phosphorus level of a normal population of subjects. A normal population of subjects can be, for example, a population of healthy subjects. An elevated level of phosphorus in a subject can be higher, substantially higher, or significantly higher than a reference or baseline value for phosphorus. Reduction in a phosphorus level of a subject can result in the phosphorus level of a subject being at or near a reference or baseline value for phosphorus. A normal human subject can have a phosphate level of about 2.4 up to about 4.1 mg/dL (see e.g., NIH MedLine). For example, a normal human subject can have a phosphate level of about 3.5 mg/dL (see e.g., Giovannucci et al. Cancer Res 58, 442-447; Health Professionals Follow-up Study). The reference or baseline value can be a phosphorus level (or average of phosphorus levels) of the same subject at an earlier time period (e.g., a phosphorus level of the subject at a time when the subject was healthy or not diagnosed with a phosphorus or FGF23-related condition).

Administration of an iron carbohydrate complex can decrease a phosphorus level in a subject by at least about 10%. For example, administration of an iron carbohydrate complex can decrease a phosphorus level in a subject by at least about 20%, at least about 30%, at least about 40%, at least about 50%, at least about 60%, at least about 70%, at least about 80%, at least about 90%, at least about 100%, or more. As another example, administration of an iron carbohydrate complex can decrease a phosphorus level in a subject by at least about 150%, at least about 200%, at least about 250%, at least about 300%, at least about 350%, at least about 400%, at least about 450%, at least about 500%, or more. Recitation of the above values includes ranges beginning or ending at each of the above values.

Administration of an iron carbohydrate complex can increase fractional excretion of phosphate in a subject by at least about 1%. For example, administration of an iron carbohydrate complex can increase fractional excretion of phosphate in a subject by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 40%, at least about 50%, at least about 60%, at least about 70%, at least about 80%, at least about 90%, at least about 100%, or more. As another example, administration of an iron carbohydrate complex can increase fractional excretion of phosphate in a subject by at least about 150%, at least about 200%, at least about 250%, at least about 300%, at least about 350%, at least about 400%, at least about 450%, at least about 500%, or more. Recitation of the above values includes ranges beginning or ending at each of the above values.

Administration of an iron carbohydrate complex can decrease phosphorus levels in a subject by at least about 0.01 mg/dL. For example, administration of an iron carbohydrate complex can decrease phosphorus levels in a subject by at least about 0.05 mg/dL, at least about 0.1 mg/dL, at least about 0.2 mg/dL, at least about 0.3 mg/dL, at least about 0.4 mg/dL, at least about 0.5 mg/dL, at least about 0.6 mg/dL, at least about 0.7 mg/dL, at least about 0.8 mg/dL, at least about 0.9 mg/dL, at least about 1.0 mg/dL, at least about 1.1 mg/dL, at least about 1.2 mg/dL, at least about 1.3 mg/dL, at least about 1.4 mg/dL, at least about 1.5 mg/dL, at least about 1.6 mg/dL, at least about 1.7 mg/dL, at least about 1.8 mg/dL, at least about 1.9 mg/dL, at least about 2.0 mg/dL, at least about 2.1 mg/dL, at least about 2.2 mg/dL, at least about 2.3 mg/dL, at least about 2.4 mg/dL, at least about 2.5 mg/dL, at least about 2.6 mg/dL, at least about 2.7 mg/dL, at least about 2.8 mg/dL, at least about 2.9 mg/dL, at least about 3.0 mg/dL, at least about 3.1 mg/dL, at least about 3.2 mg/dL, at least about 3.3 mg/dL, at least about 3.4 mg/dL, at least about 3.5 mg/dL, or more. Recitation of the above values includes ranges beginning or ending at each of the above values.

The level of phosphorus reduction can be modulated through choice of iron carbohydrate complex. As shown herein, an iron carboxymaltose can more significantly reduce phosphorus as compared to an iron dextran or an iron sucrose. Similarly, an iron dextran can more significantly reduce phosphorus as compared to an iron sucrose. Such differential modulation of phosphorus decrease can be useful according to a subjects diagnosed phosphorus level.

For example, an iron carboxymaltose can reduce phosphorus levels in a subject at least about 2-fold more (e.g., at least about 3-fold, at least about 4-fold, at least about 5-fold, at least about 6-fold, at least about 7-fold, at least about 8-fold, at least about 9-fold, or at least about 10-fold more) than an iron sucrose can reduce phosphorus levels in the same or similar subject.

As another example, an iron carboxymaltose can reduce phosphorus levels in a subject at least about 10-fold more (e.g., at least about 20-fold, at least about 30-fold, at least about 40-fold, at least about 50-fold, at least about 60-fold, at least about 70-fold, at least about 80-fold, at least about 90-fold, or at least about 100-fold more) than an iron dextran can reduce phosphorus levels in the same or similar subject. Recitation of the above values includes ranges beginning or ending at each of the above values.

An iron carboxymaltose can reduce phosphorus levels in a subject by at least about 0.1 mg/dL. For example, an iron carboxymaltose can reduce phosphorus levels in a subject by at least about 0.2 mg/dL, at least about 0.3 mg/dL, at least about 0.4 mg/dL, at least about 0.5 mg/dL, at least about 0.6 mg/dL, at least about 0.7 mg/dL, at least about 0.8 mg/dL, at least about 0.9 mg/dL, at least about 1.0 mg/dL, at least about 1.1 mg/dL, at least about 1.2 mg/dL, at least about 1.3 mg/dL, at least about 1.4 mg/dL, or at least about 1.5 mg/dL, or more. Recitation of the above values includes ranges beginning or ending at each of the above values.

As another example, an iron sucrose can reduce phosphorus levels in a subject at least about 2-fold more (e.g., at least about 3-fold, at least about 4-fold, at least about 5-fold, at least about 6-fold, at least about 7-fold, at least about 8-fold, at least about 9-fold, or at least about 10-fold more) than an iron dextran can reduce phosphorus levels in the same or similar subject. Recitation of the above values includes ranges beginning or ending at each of the above values.

An iron sucrose can reduce phosphorus levels in a subject at least about at least about 0.01 mg/dL. For example, an iron sucrose can reduce phosphorus levels in a subject at least about at least about 0.02 mg/dL, at least about 0.03 mg/dL, at least about 0.04 mg/dL, at least about 0.05 mg/dL, at least about 0.06 mg/dL, at least about 0.07 mg/dL, at least about 0.08 mg/dL, at least about 0.09 mg/dL, at least about 0.1 mg/dL, at least about 0.2 mg/dL, at least about 0.3 mg/dL, at least about 0.4 mg/dL, at least about 0.5 mg/dL, or at least about 0.6 mg/dL, or more. Recitation of the above values includes ranges beginning or ending at each of the above values.

An iron dextran can reduce phosphorus levels in a subject at least about at least about 0.001 mg/dL. For example, an iron sucrose can reduce phosphorus levels in a subject at least about at least about 0.005 mg/dL, at least about 0.01 mg/dL, at least about 0.15 mg/dL, at least about 0.2 mg/dL, or more. Recitation of the above values includes ranges beginning or ending at each of the above values.

The above described reduction in phosphorus levels in a subject can occur over a range of time. For example, a reduction in phosphorus level described above can occur in at least about an hour. As another example, a reduction in phosphorus level described above can occur in at least about a day (e.g., at least about two days, at least about three days, at least about four days, at least about five days, at least about six days). As another example, a reduction in phosphorus level described above can occur in at least about a week (e.g., at least about two weeks, at least about three weeks, at least about four weeks, at least about five weeks, at least about six weeks).

The above described reduction in phosphorus levels in a subject can be maintained, substantially maintained, partially maintained over a range of time. For example, a reduction in phosphorus level described above can be maintained, substantially maintained, or partially maintained for at least about an hour. As another example, a reduction in phosphorus level described above can be maintained, substantially maintained, or partially maintained for at least about a day (e.g., at least about two days, at least about three days, at least about four days, at least about five days, at least about six days). As another example, a reduction in phosphorus level described above can be maintained, substantially maintained, or partially maintained for at least about a week (e.g., at least about two weeks, at least about three weeks, at least about four weeks, at least about five weeks, at least about six weeks).

FGF23

As described herein, administration of an iron carbohydrate complex can decrease cFGF23 levels or increase iFGF23 levels in a subject. For example, administration of a ferric carboxymaltose or an iron dextran can decrease cFGF23 levels in a subject. Also as described herein, administration of an iron carbohydrate complex can increase iFGF23 levels in a subject. For example, administration of a ferric carboxymaltose can increase iFGF23 levels in a subject.

As described herein, an iron carbohydrate complex can reduce elevated cFGF23 in a subject. An elevated level of cFGF23 in a subject can be in comparison to a reference or baseline value for cFGF23. A reference or baseline value can be an average cFGF23 level of a normal population of subjects. A normal population of subjects can be, for example, a population of healthy subjects. An elevated level of cFGF23 in a subject can be higher, substantially higher, or significantly higher than a reference or baseline value for cFGF23. Reduction in an cFGF23 level of a subject can result in the cFGF23 level of a subject being at or near a reference or baseline value for cFGF23. A normal human subject can have a baseline cFGF23 level of about 48 up to about 73 relative units (RU)/mL (see e.g., Gutierrez et al. 2011 Clin J Am Soc Nephrol 6, 2871-2878). The reference or baseline value can be a cFGF23 level (or average of cFGF23 levels) of the same subject at an earlier time period (e.g., a cFGF23 level of the subject at a time when the subject was healthy or not diagnosed with a phosphorus or FGF23-related condition).

Administration of an iron carbohydrate complex can decrease cFGF23 levels in a subject by at least about 10%. For example, administration of an iron carbohydrate complex can decrease cFGF23 levels in a subject by at least about 20%, at least about 30%, at least about 40%, at least about 50%, at least about 60%, at least about 70%, at least about 80%, at least about 90%, at least about 100%, or more. As another example, administration of an iron carbohydrate complex can decrease cFGF23 levels in a subject by at least about 150%, at least about 200%, at least about 250%, at least about 300%, at least about 350%, at least about 400%, at least about 450%, at least about 500%, or more. Recitation of the above values includes ranges beginning or ending at each of the above values.

Administration of an iron carbohydrate complex can decrease cFGF23 levels in a subject by at least about 50 RU/mL. For example, administration of an iron carbohydrate complex can decrease cFGF23 levels in a subject by at least about 50 RU/mL, at least about 100 RU/mL, at least about 150 RU/mL, at least about 200 RU/mL, at least about 250 RU/mL, at least about 300 RU/mL, at least about 350 RU/mL, at least about 400 RU/mL, at least about 450 RU/mL, at least about 500 RU/mL, at least about 550 RU/mL, at least about 600 RU/mL, at least about 650 RU/mL, at least about 700 RU/mL, at least about 750 RU/mL, at least about 800 RU/mL, at least about 850 RU/mL, at least about 900 RU/mL, at least about 1,000 RU/mL, at least about 1,100 RU/mL, at least about 1,200 RU/mL, at least about 1,300 RU/mL, at least about 1,400 RU/mL, at least about 1,500 RU/mL, at least about 1,600 RU/mL, at least about 1,700 RU/mL, at least about 1,800 RU/mL, at least about 1,900 RU/mL, at least about 2,000 RU/mL, or more. As another example, administration of an iron carbohydrate complex can decrease cFGF23 levels in a subject by at least about 1,500 RU/mL, at least about 2,000 RU/mL, at least about 2,500 RU/mL, at least about 3,000 RU/mL, at least about 3,500 RU/mL, at least about 4,000 RU/mL, or more. Recitation of the above values includes ranges beginning or ending at each of the above values.

Administration of an iron carbohydrate complex can increase iFGF23 levels in a subject by at least about 10%. For example, administration of an iron carbohydrate complex can increase iFGF23 levels in a subject by at least about 20%, at least about 30%, at least about 40%, at least about 50%, at least about 60%, at least about 70%, at least about 80%, at least about 90%, at least about 100%, or more. As another example, administration of an iron carbohydrate complex can increase iFGF23 levels in a subject by at least about 150%, at least about 200%, at least about 250%, at least about 300%, at least about 350%, at least about 400%, at least about 450%, at least about 500%, or more. Recitation of the above values includes ranges beginning or ending at each of the above values.

As described herein, an iron carbohydrate complex can increase iFGF23 in a subject. An increased level of iFGF23 in a subject can be in comparison to a reference or baseline value for iFGF23. While conditions such as chronic kidney disease or damaged or degenerated heart tissue may modulate levels of iFGF23, it is thought that in most cases iFGF23 levels in such conditions are the same, about the same, or similar to reference or baseline iFGF23 values. A reference or baseline value can be an average iFGF23 level of a normal population of subjects. A normal population of subjects can be, for example, a population of healthy subjects. An increased level of iFGF23 in a subject can be higher, substantially higher, or significantly higher than a reference or baseline value for iFGF23. Increase in an iFGF23 level of a subject can result in the iFGF23 level of a subject more, substantially more, or significantly more than a reference or baseline value for iFGF23. The reference or baseline value can be an iFGF23 level (or average of iFGF23 levels) of the same subject at an earlier time period (e.g., an iFGF23 level of the subject at a time when the subject was healthy or not diagnosed with a phosphorus or FGF23-related condition).

Administration of an iron carbohydrate complex can increase iFGF23 levels in a subject by at least about 10 RU/mL. For example, administration of an iron carbohydrate complex can increase iFGF23 levels in a subject by at least about 10 RU/mL, at least about 20 RU/mL, at least about 30 RU/mL, at least about 40 RU/mL, at least about 50 RU/mL, at least about 60 RU/mL, at least about 70 RU/mL, at least about 80 RU/mL, at least about 90 RU/mL, at least about 100 RU/mL, at least about 110 RU/mL, at least about 120 RU/mL, at least about 130 RU/mL, at least about 140 RU/mL, at least about 150 RU/mL, at least about 200 RU/mL, at least about 250 RU/mL, at least about 300 RU/mL, at least about 350 RU/mL, at least about 400 RU/mL, at least about 450 RU/mL, at least about 500 RU/mL, at least about 550 RU/mL, at least about 600 RU/mL, at least about 650 RU/mL, at least about 700 RU/mL, at least about 750 RU/mL, at least about 800 RU/mL, at least about 850 RU/mL, at least about 900 RU/mL, at least about 1,000 RU/mL, or more. As another example, administration of an iron carbohydrate complex can increase iFGF23 levels in a subject by at least about 1,500 RU/mL, at least about 2,000 RU/mL, at least about 2,500 RU/mL, at least about 3,000 RU/mL, or more. Recitation of the above values includes ranges beginning or ending at each of the above values.

Administration of an iron carbohydrate complex can increase iFGF23 levels in a subject by at least about 10 pg/mL. For example, administration of an iron carbohydrate complex can increase iFGF23 levels in a subject by at least about 10 pg/mL, at least about 20 pg/mL, at least about 30 pg/mL, at least about 40 pg/mL, at least about 50 pg/mL, at least about 60 pg/mL, at least about 70 pg/mL, at least about 80 pg/mL, at least about 90 pg/mL, at least about 100 pg/mL, at least about 110 pg/mL, at least about 120 pg/mL, at least about 130 pg/mL, at least about 140 pg/mL, at least about 150 pg/mL, at least about 160 pg/mL, at least about 170 pg/mL, at least about 180 pg/mL, at least about 190 pg/mL, at least about 200 pg/mL, at least about 250 pg/mL, at least about 300 pg/mL, at least about 350 pg/mL, at least about 400 pg/mL, at least about 450 pg/mL, at least about 500 pg/mL, at least about 550 pg/mL, at least about 600 pg/mL, at least about 650 pg/mL, at least about 700 pg/mL, at least about 750 pg/mL, at least about 800 pg/mL, at least about 850 pg/mL, at least about 900 pg/mL, at least about 1,000 pg/mL, or more. As another example, administration of an iron carbohydrate complex can increase iFGF23 levels in a subject by at least about 1,500 pg/mL, at least about 2,000 pg/mL, at least about 2,500 pg/mL, at least about 3,000 pg/mL, or more. Recitation of the above values includes ranges beginning or ending at each of the above values.

The level of cFGF23 reduction or iFGF23 increase can be modulated through choice of iron carbohydrate complex. As shown herein, an iron carboxymaltose or iron dextran can more significantly reduce cFGF23 as compared to an iron sucrose. Also shown herein, an iron carboxymaltose can more significantly increase iFGF23 as compared to an iron dextran or an iron sucrose. Such differential modulation of FGF23 can be useful as described herein.

For example, an iron carboxymaltose or an iron dextran can reduce cFGF23 levels in a subject at least about at least about 0.25-fold more (e.g., at least about 0.5-fold, at least about 0.75-fold, at least about 1-fold, at least about 2-fold, at least about 3-fold, at least about 4-fold, or at least about 5-fold, or more) than an iron sucrose can reduce cFGF23 levels in the same or similar subject. Recitation of the above values includes ranges beginning or ending at each of the above values.

As another example, an iron carboxymaltose can increase iFGF23 levels in a subject at least about at least about 0.25-fold more (e.g., at least about 0.5-fold, at least about 0.75-fold, at least about 1-fold, at least about 2-fold, at least about 3-fold, at least about 4-fold, or at least about 5-fold, or more), whereas neither an iron dextran or an iron sucrose can significantly increase iFGF23 levels in the same or similar subject. Recitation of the above values includes ranges beginning or ending at each of the above values.

The above described reduction in cFGF23 levels or increase in iFGF23 levels in a subject can occur over a range of time. For example, a reduction in cFGF23 level or increase in iFGF23 level described herein can occur in at least about an hour. As another example, a reduction in cFGF23 level or increase in iFGF23 level described herein can occur in at least about a day (e.g., at least about two days, at least about three days, at least about four days, at least about five days, at least about six days). As another example, a reduction in cFGF23 level or increase in iFGF23 level described herein can occur in at least about a week (e.g., at least about two weeks, at least about three weeks, at least about four weeks, at least about five weeks, at least about six weeks).

The above described reduction in cFGF23 levels or increase in iFGF23 levels in a subject can be maintained, substantially maintained, or partially maintained over a range of time. For example, a reduction in cFGF23 level or increase in iFGF23 level described herein can be maintained, substantially maintained, or partially maintained for at least about an hour. As another example, a reduction in FGF23 level or increase in iFGF23 level described herein can be maintained, substantially maintained, or partially maintained for at least about a day (e.g., at least about two days, at least about three days, at least about four days, at least about five days, at least about six days). As another example, a reduction in FGF23 level or increase in iFGF23 level described herein can be maintained, substantially maintained, or partially maintained for at least about a week (e.g., at least about two weeks, at least about three weeks, at least about four weeks, at least about five weeks, at least about six weeks).

Processes for determining cFGF23 level or iFGF23 level of a subject are described herein (see e.g., Example 1-2) and known in the art. For example, levels of cFGF23 can be assessed using a sandwich ELISA (Immutopics, San Clemente, Calif.) or levels of iFGF23 can be assessed using a sandwich ELISA (Kainos Laboratories, Tokio, Japan). Except as otherwise noted herein, therefore, the process of the present disclosure can be carried out in accordance with such conventional processes.

Iron Carbohydrate Complex

Iron carbohydrate complexes are commercially available, or have well known syntheses (see e.g., Andreasen and Christensen 2001, Geisser et al. 1992 Structure/histotoxicity relationship of parenteral iron preparations. Arzneimittelforschung 42: 1439-1452; Groman and Josephson 1990, Groman et al. 1989). Examples of iron carbohydrate complexes include iron monosaccharide complexes, iron disaccharide complexes, iron oligosaccharide complexes, and iron polysaccharide complexes, such as: iron carboxymaltose, iron sucrose, iron polyisomaltose, iron dextran, iron polymaltose, iron dextrin, iron gluconate, iron sorbitol, iron hydrogenated dextran, which may be further complexed with other compounds, such as sorbitol, citric acid and gluconic acid (for example iron dextrin-sorbitol-citric acid complex and iron sucrose-gluconic acid complex), and mixtures thereof. A maltose is a disaccharide with a α-(1-4)-linkage between two glucose units. One example of a maltose-containing iron carbohydrate complex is ferric carboxymaltose (e.g., Ferinject®). An isomaltose is a disaccharide similar to maltose, but with a α-(1-6)-linkage between two glucose units instead of an α-(1-4)-linkage. One example of an iron polyisomaltose complex is an iron isomaltoside (e.g., Monofer®), where the carbohydrate component is a pure linear chemical structure of repeating α1-6 linked glucose units. In contrast, a dextran is a branched glucan with straight chains having α1-6 glycosidic linkages and branches beginning from α1-3 linkages.

Currently available parenteral iron formulations approved for use in the U.S. include iron dextran (e.g., InFed, Dexferrum, Cosmofer), sodium ferric gluconate complex in sucrose (Ferrlecit), and iron sucrose (Venofer).

An iron carbohydrate complex can be as described in U.S. Pat. No. 6,960,571, issued 1 Nov. 2005; U.S. Pat. No. 7,754,702, issued 13 Jul. 2010; International Patent Application No. WO 2007/081744, published 19 Jul. 2007; US Patent Application Publication No. 2010/0266644, published 21 Oct. 2010; each of which is incorporated herein by reference in its entirety.

An iron carbohydrate complex can be a maltose-containing iron carboxymaltose complex. An iron carbohydrate complex can be an iron carboxymaltose complex. An iron carboxymaltose complex can be according to U.S. Pat. No. 7,754,702, issued 13 Jul. 2010, or US Patent Application Publication No. 2010/0266644, published 21 Oct. 2010, each incorporated by reference herein in its entirety. An example of an iron carboxymaltose complex is polynuclear iron (III)-hydroxide 4(R)-(poly-(1→4)-O-α-glucopyranosyl)-oxy-2(R),3(S),5(R),6-tetrahydroxy-hexanoate (“FCM”). FCM is a Type I polynuclear iron (III) hydroxide carbohydrate complex that can be administered as parenteral iron replacement therapy for the treatment of various anemia-related conditions as well as other iron-metabolism related conditions. FCM can be represented by the chemical formula: [FeOx(OH)y(H2O)z]n [{(C6H10O5)m (C6H12O7)}l]k, where n is about 103, m is about 8, l is about 11, and k is about 4). The molecular weight of FCM is about 150,000 Da.

The degradation rate and physicochemical characteristics of FCM make it an efficient means of parenteral iron delivery to the body stores. It is more efficient and less toxic than the lower molecular weight complexes such as iron sorbitol/citrate complex, and does not have the same limitations of high pH and osmolarity that leads to dosage and administration rate limitations in the case of, for example, iron sucrose and iron gluconate.

FCM generally does not contain dextran and does not substantially react with dextran antibodies; therefore, the risk of anaphylactoid/hypersensitivity reactions is very low compared to iron dextran. FCM has a nearly neutral pH (5.0 to 7.0) and physiological osmolarity, which makes it possible to administer higher single unit doses over shorter time periods than other iron-carbohydrate complexes. FCM can mimic physiologically occurring ferritin. FCM is metabolized by the glycolytic pathway. Like iron dextran, FCM is more stable than iron gluconate or sucrose. FCM produces a slow and competitive delivery of the complexed iron to endogenous iron binding sites resulting in an acute toxicity one-fifth that of iron sucrose.

After intravenous administration, FCM is mainly found in the liver, spleen, and bone marrow. Pharmacokinetic studies using positron emission tomography have demonstrated a fast initial elimination of radioactively labeled iron (Fe) ⁵²Fe/⁵⁹Fe FCM from the blood, with rapid transfer to the bone marrow and rapid deposition in the liver and spleen. See e.g., Beshara et al. (2003) Br J Haematol 2003; 120(5): 853-859. Eight hours after administration, 5 to 20% of the injected amount was observed to be still in the blood, compared with 2 to 13% for iron sucrose. The projected calculated terminal half-life (t_{1/2}) was approximately 16 hours, compared to 3 to 4 days for iron dextran and 6 hours for iron sucrose.

Single-dose toxicity studies have demonstrated safety and tolerance in rodents and dogs of intravenous doses of FCM up to 60 times more than the equivalent of an intravenous infusion of 1,000 mg iron once weekly in humans. Pre-clinical studies in dogs and rats administered FCM in cumulative doses up to 117 mg iron/kg body weight over 13 weeks showed no observed adverse effect level in dose-related clinical signs of iron accumulation in the liver, spleen, and kidneys. No treatment-related local tissue irritation was observed in intra-arterial, perivenous, or intravenous tolerance studies in the rabbit. In vitro and in vivo mutagenicity tests provided no evidence that FCM is clastogenic, mutagenic, or causes chromosomal damage or bone marrow cell toxicity. There were no specific responses to FCM in a dextran antigenicity test.

An isomaltose is a disaccharide similar to maltose, but with a α-(1-6)-linkage between two glucose units instead of an α-(1-4)-linkage. One example of an iron polyisomaltose complex is an iron isomaltoside (e.g., Monofer®), where the carbohydrate component is a pure linear chemical structure of repeating α1-6 linked glucose units. In contrast, a dextran is a branched glucan with straight chains having α1-6 glycosidic linkages and branches beginning from α1-3 linkages.

Formulation

In many cases, a single unit dose of iron carbohydrate complex may be delivered as a simple composition comprising the iron complex and the buffer in which it is dissolved. However, other products may be added, if desired, for example, to maximize iron delivery, preservation, or to optimize a particular method of delivery.

The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

A pharmaceutically acceptable carrier includes any and all solvents, dispersion media, coatings, antibacterial and anti-fungal agents, isotonic and absorption delaying agents, and the like, compatible with pharmaceutical administration (see e.g., Banker, Modern Pharmaceutics, Drugs and the Pharmaceutical Sciences, 4th ed. (2002) ISBN 0824706749; Remington The Science and Practice of Pharmacy, 21st ed. (2005) ISBN 0781746736). Preferred examples of such carriers or diluents include, but are not limited to, water, saline, Finger's solutions and dextrose solution. Supplementary active compounds can also be incorporated into the compositions. For intravenous administration, the iron carbohydrate complex is preferably diluted in normal saline to approximately 2-5 mg/ml. The volume of the pharmaceutical solution is based on the safe volume for the individual patient, as determined by a medical professional.

An iron complex composition of the invention for administration is formulated to be compatible with the intended route of administration, such as intravenous injection. Solutions and suspensions used for parenteral, intradermal or subcutaneous application can include a sterile diluent, such as water for injection, saline solution, polyethylene glycols, glycerine, propylene glycol or other synthetic solvents; antibacterial agents such as benzyl alcohol or methyl parabens; antioxidants such as ascorbic acid or sodium bisulfite; buffers such as acetates, citrates or phosphates, and agents for the adjustment of tonicity such as sodium chloride or dextrose. The pH can be adjusted with acids or bases, such as hydrochloric acid or sodium hydroxide. Preparations can be enclosed in ampules, disposable syringes or multiple dose vials made of glass or plastic.

Pharmaceutical compositions suitable for injection include sterile aqueous solutions or dispersions for the extemporaneous preparation of sterile injectable solutions or dispersion. For intravenous administration, suitable carriers include physiological saline, bacteriostatic water, Cremophor EL™ (BASF; Parsippany, N.J.) or phosphate buffered saline (PBS). The composition must be sterile and should be fluid so as to be administered using a syringe. Such compositions should be stable during manufacture and storage and must be preserved against contamination from microorganisms, such as bacteria and fungi. The carrier can be a dispersion medium containing, for example, water, polyol (such as glycerol, propylene glycol, and liquid polyethylene glycol), and other compatible, suitable mixtures. Various antibacterial and anti-fungal agents, for example, parabens, chlorobutanol, phenol, ascorbic acid, and thimerosal, can contain microorganism contamination. Isotonic agents such as sugars, polyalcohols, such as manitol, sorbitol, and sodium chloride can be included in the composition. Compositions that can delay absorption include agents such as aluminum monostearate and gelatin.

Sterile injectable solutions can be prepared by incorporating an iron complex in the required amount in an appropriate solvent with a single or combination of ingredients as required, followed by sterilization. Methods of preparation of sterile solids for the preparation of sterile injectable solutions include vacuum drying and freeze-drying to yield a solid containing the iron complex and any other desired ingredient.

Active compounds may be prepared with carriers that protect the compound against rapid elimination from the body, such as a controlled release formulation, including implants and microencapsulated delivery systems. Biodegradable or biocompatible polymers can be used, such as ethylene vinyl acetate, polyanhydrides, polyglycolic acid, collagen, polyorthoesters, and polylactic acid. Such materials can be obtained commercially from ALZA Corporation (Mountain View, Calif.) and NOVA Pharmaceuticals, Inc. (Lake Elsinore, Calif.), or prepared by one of skill in the art.

A preferred pharmaceutical composition for use in the methods described herein contains FCM as the active pharmaceutical ingredient (API) with about 28% weight to weight (m/m) of iron, equivalent to about 53% m/m iron (III)-hydroxide, about 37% m/m of ligand, ≦6% m/m of NaCl, and ≦10% m/m of water.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating a disease or disorder associated with increased phosphorus or increased cFGF23 levels in a subject in need by administration of a therapeutically effective amount of an iron carbohydrate complex, so as to reduce phosphorus levels, reduce cFGF23 levels, or increase iFGF23 levels. Also provided is a process of reducing phosphate, reducing cFGF23, or increasing iFGF23 in a subject in need by administration of a therapeutically effective amount of an iron carbohydrate complex, so as to reduce phosphorus levels, reduce cFGF23 levels, or increase iFGF23 levels.

Administration of an iron carbohydrate complex can be as described in U.S. Pat. No. 6,960,571, issued 1 Nov. 2005; U.S. Pat. No. 7,754,702, issued 13 Jul. 2010; International Patent Application No. WO 2007/081744, published 19 Jul. 2007; US Patent Application Publication No. 2010/0266644, published 21 Oct. 2010; each of which is incorporated herein by reference in its entirety.

Methods described herein are generally performed on a subject in need thereof. A subject in need of the therapeutic methods described herein can be a subject having, diagnosed with, suspected of having, or at risk for developing increased phosphorus or increased cFGF23 levels. For example, increased phosphorus or increased cFGF23 levels can be associated with chronic kidney disease. A subject in need of treatment according to the methods described herein can be diagnosed with chronic kidney disease. For example, increased phosphorus or increased cFGF23 levels can be associated with hyperphosphatemia. A subject in need of treatment according to the methods described herein can be diagnosed with hyperphosphatemia. As another example, increased phosphorus or increased cFGF23 levels can be associated with damaged or degenerated heart tissue (i.e., heart tissue which exhibits a pathological condition). Causes of heart tissue damage or degeneration include, but are not limited to, chronic heart damage, chronic heart failure, damage resulting from injury or trauma, damage resulting from a cardiotoxin (e.g., FGF23), damage from radiation or oxidative free radicals, damage resulting from decreased blood flow, and myocardial infarction (such as a heart attack). A subject in need of treatment according to the methods described herein can be diagnosed with degenerated heart tissue resulting from a myocardial infarction or heart failure.

A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, guinea pigs, and chickens, and humans. For example, the subject can be a human subject.

Generally, a safe and effective amount of an iron carbohydrate complex is, for example, that amount that would cause the desired therapeutic effect (e.g., reduction in phosphate levels, reduction in cFGF23 levels, or increase in IFGF23 levels) in a subject while minimizing undesired side effects. The dosage regimen will be determined by skilled clinicians, based on factors such as the exact nature of the condition being treated, the severity of the condition, the age and general physical condition of the patient, and so on. Generally, treatment-emergent adverse events will occur in less than about 5% of treated patients. For example, treatment-emergent adverse events will occur in less than 4% or 3% of treated patients. Preferably, treatment-emergent adverse events will occur in less than about 2% of treated patients.

For example, minimized undesirable side effects can include those related to hypersensitivity reactions, sometimes classified as sudden onset closely related to the time of dosing, including hypotension, bronchospasm, layngospasm, angioedema or uticaria or several of these together. Hypersensitivity reactions are reported with all current intravenous iron products independent of dose (see generally, Bailie et al. 2005 Nephrol Dial Transplant 20(7), 1443-1449). As another example, minimized undesirable side effects can include those related to labile iron reactions, sometimes classified as nausea, vomiting, cramps, back pain, chest pain, and/or hypotension. Labile iron reactions are more common with iron sucrose, iron gluconate, and iron dextran when doses are large and given fast.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of an iron carbohydrate complex can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to reduce phosphorus levels, reduce cFGF23 levels, or increase iFGF23 levels in a subject.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the patient; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Administration of an iron carbohydrate complex can occur as a single event or over a time course of treatment. For example, an iron carbohydrate complex can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for diseases or disorders characterized by increased levels of phosphorus or increased levels of cFGF23.

An iron carbohydrate complex can be administered simultaneously or sequentially with another agent, such as an antibiotic, an antiinflammatory, or another agent. For example, an iron carbohydrate complex can be administered simultaneously with another agent, such as an antibiotic or an antiinflammatory. Simultaneous administration can occur through administration of separate compositions, each containing one or more of an iron carbohydrate complex, an antibiotic, an antiinflammatory, or another agent. Simultaneous administration can occur through administration of one composition containing two or more of a an iron carbohydrate complex, an antibiotic, an antiinflammatory, or another agent. An iron carbohydrate complex can be administered sequentially with an antibiotic, an antiinflammatory, or another agent. For example, an iron carbohydrate complex can be administered before or after administration of an antibiotic, an antiinflammatory, or another agent.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to an iron carbohydrate complex or reagents for detection of phosphate, cFGF23, or iFGF23. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### VIT-23

The following example describes a randomized, active controlled trial of subjects with iron deficiency anemia of any etiology (but mainly women with heavy uterine bleeding) who received a single dose of 1000 mg of either ferric carboxymaltose (FCM) or Iron Dextran on Day 0 and were then assessed at subsequent study visits on Days 1, 7, 14, and 35. An objective was to assess the safety of FCM or iron dextran and explore the mechanism of hypophosphatemia following administration of FCM or that of an equal dose of iron dextran.

VIT-23 was a Phase IIa, open label, multicenter, randomized study that compared the safety and explored the mechanism of hypophosphatemia with an investigational IV iron (ferric carboxymaltose) compared to an equal total dose of iron dextran in treating iron deficient women with Heavy Uterine Bleeding (HUB). Subjects with a hemoglobin (Hgb) that meets inclusion requirements and no exclusionary criteria will be offered participation in this 5-6 week study.

During the Treatment Phase, all subjects received a maximum dose of 1000 mg of IV iron. Group A subjects received an infusion of FCM (15 mg/kg up to a maximum of 1000 mg) IV diluted in 250 cc normal saline solution (NSS) and administered over 15 minutes on Day 0. Group B subjects received IV iron dextran on Day 0 as follows: a test dose of 25 mg administered slowly over 5 minutes and the subject observed for 15 minutes to 1 hour. If no reaction occurred, the remainder of the dose (up to 15 mg/kg or 1000 mg including the test dose) was given as per Investigator. The infusion was given only when resuscitative techniques for the treatment of anaphylactic and anaphylactoid reactions were readily available.

Sample size was twenty subjects per treatment group for a total of 40 evaluable subjects (i.e., subjects for whom interpretable blood and urine samples are provided at Days 0, 1, 7, 14 and 35). In prior studies of patients treated with FCM due to iron deficiency anemia secondary to heavy uterine bleeding (1VIT04002/1VIT04003), phosphate was measured at baseline and days 7, 14, 28, and 42; and the mean change in phosphate was 1.9 mg/dL for patients receiving FCM (N=228), which corresponded to 2.5 standard deviations. A sample size of 20 subjects would therefore provide >90% power for a 0.8 standard deviation shift from baseline. If the mean change in phosphate for the iron dextran group was less than half that of FCM, 20 subjects per group would also provide >90% power to detect a statistically significant difference between the FCM and iron dextran groups. Thus, the sample size should have provided adequate power to assess clinically significant urine or blood biomarkers underlying the mechanism of the phosphate changes. Similarly, the sample size should have been adequate to detect changes in FGF-23 reported in other trials.

Subject inclusion criteria included:
Female subjects≧18 years of age, able to give informed consent to the study;
History of Heavy Uterine Bleeding within the past 6 months, defined as any one of (a) inability to control flow with tampons alone (b) use of more than 12 pads per period or 4 tampons per day, unless subject is unusually fastidious (c) passage of clots, especially if they are larger than approximately 2 cm (nickel size) in diameter or if they persist after the first day or (d) period duration exceeding 7 days;
Screening Visit central laboratory Hgb<12 g/dL;
Screening Visit ferritin≦100 ng/mL or ≦300 when TSAT is ≦30%; and
Demonstrate the ability to understand the requirements of the study, willingness to abide by study restrictions and to return for the required assessments.

Subject exclusion criteria included:
Known hypersensitivity reaction to any component of ferric carboxymaltose or iron dextran;
Previously randomized in a clinical study of FCM (VIT-45);
Requires dialysis for treatment of chronic kidney disease;
Chronic kidney disease, marked by estimated glomerular filtration rate<60 ml/min/1.73 m²;
Previous kidney transplant;
History of primary hypophosphatemic disorder (for example X-linked hypophosphatemia);
Hypophosphatemia<2.6 mg/dl;
No evidence of iron deficiency;
During the 10 day period prior to screening has been treated with intravenous iron;
During the 30 day period prior to screening or during the study period has or will be treated with erythropoiesis stimulating agents (ESA) in a regimen that is off label;
During the 30 day period prior to screening or during the study period has or will be treated with a red blood cell transfusion, radiotherapy and/or chemotherapy;
During the 30 day period prior to screening or during the study period has or will require a surgical procedure that necessitates general anesthesia;
Any non-viral infection;
AST or ALT at screen 1, as determined by central labs, greater than 1.5 times the upper limit of normal;
Known positive hepatitis with evidence of active disease;
Received an investigational drug within 30 days of Screening;
Alcohol or drug abuse within the past 6 months;
Hemochromatosis or other iron storage disorders;
Malignancy history within the past 5 years other than basal or squamous cell skin cancer;
Any other laboratory abnormality, medical condition or psychiatric disorders which in the opinion of the investigator would put the subject's disease management at risk or may result in the subject being unable to comply with study requirements;
Pregnant or sexually-active female subjects who are of childbearing potential and who are not willing to use an acceptable form of contraception (s/p tubal ligation or otherwise incapable of pregnancy, hormonal contraceptives, spermicide plus barrier or intrauterine device);
Untreated primary hyperparathyroidism; or
Untreated gastrointestinal malabsorption (e.g., sprue).

Primary Endpoints included changes in blood and urine markers of phosphate and bone metabolism following IV iron administration. Blood markers include (all performed in morning after 10 hr fast): Serum Phosphate; Serum Potassium; Plasma Fibroblast growth factor 23 via 2^{nd} generation Immutopics C-terminal assay; Serum Vitamin D; 1, 25 dihydroxy Vitamin D (1,25 [OH]₂D); 25 hydroxy Vitamin D (250H-D); Serum Osteocalcin; Serum Bone-specific Alkaline phosphatase; Plasma intact Parathyroid hormone; Serum creatinine; Hepcidin; Reticulocyte count; Hemoglobin; Ferritin; Transferrin Saturation (TSAT); and Sample for archive. Urine markers (collected over a 3 hour period in the morning) (ratio of phosphate to creatinine determined to offset inter-patient variability in urine concentration) include: Phosphate; Creatinine; Calcium; Amino acids; Glucose; Albumin; Deoxypyridinoline cross link (Dpd)—a collagen type I cross link that indicates bone resorption; Beta-2-microglobulin; and Sample for archive.

Calculated values include (a) Tubular reabsorption of phosphate and (b) Fractional excretion of phosphate.

Secondary Endpoints included Safety and Efficacy. Safety Secondary Endpoints included (a) incidence, severity, and seriousness of adverse events, overall and related from Day 0 through end of study (Day 35) or 30 days after the last dose of study drug whichever is longer; and (b) incidence of treatment-emergent abnormal clinical laboratory values. Efficacy Secondary Endpoints included (a) the proportion of subjects achieving a hemoglobin increase 2 g/dL anytime between baseline and end of study or time of intervention and (b) change from baseline to highest hemoglobin, ferritin, and TSAT anytime between baseline and end of study or time of intervention.

All subjects had laboratory assessments at Screening, Day 0, Day 1 (24 hrs), Day 7, Day 14, and Day 35. Any subject with a change in phosphate value after Day 0 had additional phosphate measurements at 14 day intervals (following Day 35) until the level returned to normal (as defined by the laboratory reference range). Safety evaluation for all subjects included adverse events, physical examination, including vital signs, and laboratory assessments. Three hour urine collections was also performed on Day 0, Day 1 (24 hrs), Day 7, Day 14, and Day 35. Any subject who withdrew from the study will receive a follow up phone call 30 days after they received study drug.

Study duration was 5-6 weeks across up to 10 study sites.

TABLE 1
Results of VIT-23 Study

analyte (unit)

Phosphorus

| (mg/dL) | Visit | Statistic | Group A: FCM | Group B: Iron Dextran |
|---|---|---|---|---|
| Day 7 | Baseline | n | 31 | 36 |

| | | | | |
|---|---|---|---|---|
| Mean(SD) | 3.39 | (0.404) | 3.63 | (0.531) |

| | | | |
|---|---|---|---|
| Visit | n | 31 | 36 |

| | | | | |
|---|---|---|---|---|
| Mean(SD) | 2.51 | (0.860) | 3.55 | (0.541) |

| | | | |
|---|---|---|---|
| Change to Visit | n | 31 | 36 |

| | | | | |
|---|---|---|---|---|
| Mean(SD) | −0.87 | (0.845) | −0.07 | (0.475) |

| | | | | |
|---|---|---|---|---|
| | | Median | −0.8 | −0.1 |
| | | Min, Max | −2.3, 0.7 | −0.9, 1.2 |
| Day 14 | Baseline | n | 33 | 27 |

| | | | | |
|---|---|---|---|---|
| Mean(SD) | 3.33 | (0.432) | 3.68 | (0.532) |

| | | | |
|---|---|---|---|
| Visit | n | 33 | 27 |

| | | | | |
|---|---|---|---|---|
| Mean(SD) | 3.42 | (0.956) | 3.58 | (0.517) |

| | | | |
|---|---|---|---|
| Change to Visit | n | 33 | 27 |

| | | | | |
|---|---|---|---|---|
| Mean(SD) | −0.92 | (0.924) | −0.06 | (0.632) |

| | | | | |
|---|---|---|---|---|
| | | Median | −0.7 | 0.1 |
| | | Min, Max | −3.5, 0.7 | −1.5, 1.4 |
| Day 35 | Baseline | n | 31 | 26 |

| | | | | |
|---|---|---|---|---|
| Mean(SD) | 3.29 | (0.412) | 3.64 | (0.545) |

| | | | |
|---|---|---|---|
| Visit | n | 31 | 26 |

| | | | | |
|---|---|---|---|---|
| Mean(SD) | 3.92 | (0.937) | 3.55 | (0.475) |

| | | | |
|---|---|---|---|
| Change to Visit | n | 31 | 26 |

| | | | | |
|---|---|---|---|---|
| Mean(SD) | −0.38 | (0.878) | −0.08 | (0.633) |

| | | |
|---|---|---|
| Median | −0.2 | −0.1 |
| Min, Max | −1.9, 1.0 | −1.3, 1.3 |

| Analyte (unit) | | | Group A: FCM | | | Group B: Iron Dextran | | | |
|---|---|---|---|---|---|---|---|---|---|
| FGF-23 | | | | | Wilxcox signed- | | | Wilxcox signed- | Between |
| C-Terminal, | | | | | rank test | | | rank test | Group |
| EDTApl-RUO | | | | | p-value for | | | p-value for | Wilxcox |
| (RU/mL) | Visit | Statisic | N = 17 | | change | N = 32 | | change | p-value |
| Day 1 | | N | 16 | | | 20 | | | |
| | Baseline | Mean(SD) | 825.70 | (690.630) | | 867.69 | (871.534) | | |
| | Visit | Mean(SD) | 176.80 | (51.088) | | 151.60 | (83.251) | | |
| | Change to Visit | Mean(SD) | −648.30 | (669.386) | <0.001*** | −716.03 | (883.032) | <0.001*** | 0.911 |
| Day 7 | | N | 12 | | | 18 | | | |
| | Baseline | Mean(SD) | 962.85 | (778.214) | | 797.54 | (890.053) | | |
| | Visit | Mean(SD) | 165.23 | (143.863) | | 114.13 | (82.630) | | |
| | Change to Visit | Mean(SD) | −699.73 | (902.401) | 0.001*** | −682.42 | (864.517) | <0.001*** | 0.849 |
| Day 14 | | N | 16 | | | 18 | | | |
| | Baseline | Mean(SD) | 754.70 | (703.180) | | 841.15 | (979.393) | | |
| | Visit | Mean(SD) | 170.12 | (329.514) | | 107.39 | (74.645) | | |
| | Change to Visit | Mean(SD) | −584.59 | (737.457) | 0.001*** | −733.96 | (831.400) | <0.001*** | 0.641 |
| Day 35 | | N | 13 | | | 30 | | | |
| | Baseline | Mean(SD) | 863.46 | (732.086) | | 852.29 | (876.279) | | |
| | Visit | Mean(SD) | 141.56 | (303.534) | | 360.71 | (240.130) | | |
| | Change to Visit | Mean(SD) | −692.29 | (802.560) | 0.005** | −683.57 | (710.286) | <0.001*** | 0.839 |
| Minimum | | N | 17 | | | 32 | | | |
| | Baseline | Mean(SD) | 782.30 | (691.179) | | 836.87 | (847.147) | | |
| | Visit | Mean(SD) | 98.73 | (24.084) | | 94.00 | (62.116) | | |
| | Change to Visit | Mean(SD) | −684.57 | (681.882) | <0.001*** | −732.67 | (801.859) | <0.001*** | 0.966 |

The VIT-23 study showed the values of phosphorus and FGF-23 at each study visit and the change with respect to baseline (see also Example 2, VIT30 study). The “baseline” was the value at Day 0 prior to administration of any study drug. The value of “n” for the baseline measurement may have be different at each study visit because the baseline values were assessed only for those individuals with data at the given visit.

### Example 2

### VIT-30

The following example describes a randomized, active controlled trial of subjects with iron deficiency anemia and non-dialysis dependent chronic kidney disease and elevated cardiovascular risk who received either FCM (750 mg on Day 0 and Day 7 for a total of 1500 mg) or iron sucrose (Venofer; 200 mg on 5 visits between Days 0 and 14 for a total of 1000 mg). Study visits were on Days 0, 3, 7, 10, 14, 28, 56, and 120. A phone assessment occurred on Day 90. The primary objective was to estimate the cardiovascular safety and efficacy FCM compared to Venofer in subjects who had iron deficiency anemia (IDA) and impaired renal function.

VIT-30 was a Phase 3, multicenter, randomized, active-controlled, open-label study that compared the safety and efficacy of IV FCM vs. IV Venofer in subjects with IDA and impaired renal function.

Subjects

Subjects must have had a hemoglobin≦11.5 g/dL (based on the mean of 2 values determined by central laboratories drawn within 7 days; the 2 values being within 0.7 mg/dL of each other) and chronically impaired renal function as defined by either of the following: (1) GFR<60 ml/min/1.73 m² on 2 measurements during the screening period (using the Modification of Diet in Renal Disease [MDRD] calculation) or (2) GFR<90 ml/min/1.73 m² on 2 measurements during the screening period and either one or both of (a) Kidney damage as indicated by abnormalities in composition of urine (as documented in the subject's medical history) or (b) Elevated risk of cardiovascular disease (Category 2 or 3) based on the Framingham Model.

Subjects that met all inclusion and none of the exclusion criteria were offered participation in this approximately 4-month study. Subjects were stratified by baseline hemoglobin (≦9, 9.1 to 10.0, ≧10.1 g/dL), baseline cardiovascular risk (history of myocardial infarction, stroke, or congestive heart failure [yes/no]), erythropoietin use (yes/no), and chronic kidney disease (CKD) stage as per National Kidney Foundation Outcome Quality Initiative (K/DOQI) stage of CKD (2, 3-4, or 5), and randomized in a 1:1 ratio to receive either IV FCM or IV Venofer.

During the Treatment Phase, the FCM Group received 2 doses of FCM at 15 mg/kg to a maximum of 750 mg per dose for a maximum total dose of 1500 mg. The Venofer Group received 5 doses of Venofer, 200 mg for a total dose of 1,000 mg.

Randomized subjects returned for efficacy and safety evaluations, which included adverse events and laboratory assessments, on Days 3, 7, 11, 14, 28, and 56. Subjects may have received additional IV iron after Day 56 at the discretion of the Investigator. Subjects received either a single dose of FCM, 750 mg (15 mg/kg) if the subject was in the FCM Group or an additional 1-4 dose course of Venofer, 200 mg (200-800 mg total) if the subject was in the Venofer Group. No additional iron was administered between Day 90 and the Day 120 Visit. In addition, subjects were contacted on Day 90 and returned to the clinic on Day 120 to assess for adverse events.

TABLE 2
Number of Subjects in VIT-30. Diagnosis and Main
Criteria for Inclusion: Male or female subjects ≧18 years of age with
chronically impaired renal function, with hemoglobin ≦11.5 g/dL at
screening and ferritin ≦100 ng/mL or ≦300 ng/mL when transferrin
saturation (TSAT) was ≦30%.

| | Group A: FCM | Group B: Venofer |
|---|---|---|
| Planned | 1250 | 1250 |
| Randomized | 1290 | 1294 |
| Treated and Evaluated | 1276 | 1285 |
| for Safety | | |

Dosage

For FCM dosage, subjects received 750 mg of iron as undiluted FCM (15 mg/kg up to a maximum of 750 mg) on Days 0 and 7 (i.e., for a total of 1,500 mg) as an IV push injection at 100 mg/minute.

For Venofer, subjects received, as an IV push injection over 2 to 5 minutes, 200 mg on Days 0, 7, and 14 with 2 additional doses given: 1 between Days 0 and 7 and the other between Days 7 and 14 (i.e., for a total of 5 doses [1,000 mg]).

Duration of treatment was approximately 4 months.

Evaluations

Criteria for evaluations included efficacy and safety.

For efficacy, the primary efficacy measure was the mean change from baseline to the highest observed hemoglobin any time between baseline and end of treatment period (Day 56) or time of intervention. Supportive efficacy measures included the following: (a) proportion of subjects achieving an increase in hemoglobin of ≧1 g/dL any time between baseline and end of treatment period (Day 56) or time of intervention; (b) mean change from baseline to the highest observed ferritin any time between baseline and treatment period (Day 56) or time of intervention; (c) mean change from baseline to the highest observed TSAT any time between baseline and treatment period (Day 56) or time of intervention; and (d) mean change from baseline to the pre-dosing value on Day 7 for hemoglobin, ferritin, and TSAT.

For safety, the primary measure was the proportion of subjects experiencing treatment-emergent adverse event included in the primary composite safety endpoint. Treatment-emergent events included events that started on or after the first dose of randomized treatment. The composite safety endpoint included: deaths due to any cause; nonfatal myocardial infarction; nonfatal stroke; unstable angina requiring hospitalization; congestive heart failure requiring hospitalization or medical intervention; arrhythmias; protocol-defined hypertensive events; and protocol-defined hypotensive events. These events were adjudicated in a blinded fashion. Supportive safety measures included: proportion of subjects who died (all cause mortality); time to first event comprising the primary composite safety endpoint; proportion of subjects reporting treatment-emergent adverse events, overall and related; proportion of subjects reporting treatment-emergent serious adverse events, overall and related; incidence of treatment-emergent potentially clinically significant (PCS) clinical laboratory values; and incidence of treatment-emergent PCS vital sign values.

Statistical Methods

The Safety Population consisted of all subjects who received a dose of randomized treatment. All safety analyses were performed with the Safety Population.

The primary population for evaluating all efficacy endpoints was the modified Intent-to-Treat (mITT) population, defined as subjects from the Safety Population who: received at least 1 dose of randomized study medication; had at least 1 post baseline hemoglobin assessment; had a stable (±20%) ESA for 4 weeks, which may have included a dose of zero (0), before randomization.

Efficacy Analysis

The noninferiority of FCM to Venofer for change from baseline to highest hemoglobin any time between baseline and end of treatment period (Day 56) or time of intervention was assessed with a 95% 2-sided confidence interval (CI, based on normal distribution, assuming equal variances) and a noninferiority margin of 0.2 g/dL. The hemoglobin baseline was defined as the average of the last 2 hemoglobin values from the central laboratory prior to the first dose of study drug. If only 1 centrally-measured hemoglobin value was available prior to the first dose of study drug, the single value was used for baseline. Non-inferiority was concluded if the lower limit of the 2-sided CI was ≧−0.075. This non-inferiority margin preserved >50% of the difference from oral iron observed previously.

For the proportion of subjects achieving an increase in hemoglobin of g/dL any time between baseline and end of treatment period (Day 56) or time of intervention, the treatment difference in efficacy (FCM versus Venofer) was estimated with a 95% 2-sided CI. The CI was based on the normal approximation for the binomial distribution, using the Wald continuity correction [(1/N₁+1/N₂/2], where N₁ was the number of subjects treated with FCM and N₂ was the number of subjects treated with Venofer.

The mean change from baseline to highest TSAT and ferritin was assessed for treatment group differences with the 95% 2-sided CI (based on normal distribution, assuming equal variances). Mean change from baseline to each scheduled visit was similarly assessed for hemoglobin, TSAT, ferritin, serum iron, total iron binding capacity, and unsaturated iron binding capacity. Baseline was calculated as described above. Assessments were assigned to scheduled visits as follows (if the assessment was on a dosing day for IV iron, the assessment before dosing was selected):
Day 7: Assessments on Days 4 through 10.
Day 14: Assessments on Days 11 through 21.
Day 28: Assessments on Days 22 through 35.
Day 56: Assessments on Days 50 through 63.

Safety Analysis

Safety assessments were summarized for the Safety Population unless stated otherwise. Subjects were analyzed under the treatment actually received.

Categorical endpoints were summarized with the number and percent of subjects in each treatment group. Quantitative endpoints were summarized with the mean, median, standard deviation, minimum value, and maximum value.

The difference between FCM and Venofer in the proportion of subjects experiencing the primary composite endpoint was assessed with a 95% 2-sided CI constructed with the normal approximation to the binomial with continuity correction. The CI was based on the normal approximation for the binomial distribution, using the Wald continuity correction [(1/N1+1/N2)/2], where N1 was the number of subjects treated with FCM and N2 was the number of subjects treated with Venofer. Evidence of equivalent cardiovascular risk for FCM and Venofer were provided if the 95% CI of the treatment difference included zero. The precision of the estimated treatment group difference was measured by the width of the 95% CI.

All adverse events were classified with respect to SOC and preferred term using MedDRA. No formal statistical tests were performed.

The number and percent of subjects who reported treatment-emergent adverse events were summarized for each treatment group. A treatment-emergent adverse event was an event that began after receipt of randomized treatment.

Adverse event summaries excluded preferred terms that describe asymptomatic serum ferritin, TSAT, and reticulocyte values (or changes). This approach was justified by the reporting of these values in efficacy summaries and was consistent with the protocol-defined reporting standards for hemoglobin/hematocrit and low iron indices. For the purposes of this study, non-serious anemia (hemoglobin or hematocrit below the normal range or worsened from baseline) or iron deficiency (iron indices below the normal range or worsened from baseline) were not considered adverse events. Anemia or iron deficiency was considered an end point if an intervention was required.

The adverse event profile was characterized with severity (as graded by Version 3.0 of the NCI CTCAE) and relationship (unrelated and related) to study drug. Related adverse events were events that were possibly or probably related to treatment in the Investigator's judgment. Events with unknown severity or relationship were counted as unknown.

The number and percent of subjects who reported treatment-emergent serious adverse events were similarly summarized for each treatment group. The number and percent of subjects who reported treatment-emergent adverse events resulting in discontinuation of study drug were similarly summarized for each treatment group.

The number and percent of subjects who died were summarized for each treatment group.

Time to the first adverse event comprising the primary composite safety endpoint was summarized with the Kaplan-Meier approach. Subjects who did not experience an event were censored on the date of their last documented study visit. The log-rank statistic were used to compare time-to-event curves between treatment groups and a 95% CI of the hazard ratio was calculated with a Cox proportional hazards model.

Clinical laboratory variables were presented in 2 ways. First, change from baseline to Day 7, Day 14, Day 28, and Day 56 were summarized. Baseline was defined as the last value obtained prior to the first injection of study drug. Second, the number and proportion of subjects with treatment-emergent PCS laboratory values were tabulated. Treatment-emergent PCS values were those in which the baseline value was normal and post-baseline value was abnormal (i.e., met Grade 3 or Grade 4 toxicity criteria from the National Cancer Institute Common Toxicity Criteria for Adverse Events, Version 3.0 [NCI CTCAE]).

For serum phosphate and platelet count, change from baseline to the smallest value after baseline and final value were also summarized. For subjects whose baseline value was above the lower reference range and who had at least 1 value below the lower reference range after baseline, the following were summarized: the number of days from baseline to the first value below the lower reference range and the number of days from the first value below the lower reference range until the first succeeding value within the reference range.

No formal statistical tests were performed. Laboratory values were converted to the project defined unit of measurement before analysis.

Treatment-emergent PCS vital signs were identified on dosing days for FCM and Venofer. The number and percent of subjects with PCS vital signs following planned injections of FCM and Venofer between Days 0 and 14, inclusive, were summarized. Planned injections that were delayed were included if administered before Day 56. No formal statistical tests were performed.

The Concomitant Medications World Health Organization drug dictionary was used to classify all concomitant medications with respect to the Anatomical-Therapeutic-Chemical classification (ATC) system and preferred drug name. Concomitant drug usage was summarized by ATC level 3 and preferred drug name. The summary provided the number and percent of subjects in each treatment group who received at least 1 non-study medication.

Medications received prior to the first dose of study drug were tabulated and listed separately.

The number and percent of subjects receiving EPO at baseline were summarized. For subjects receiving EPO at baseline, the dose of EPO was summarized descriptively at baseline. The dose of EPO was also summarized at Day 56 and Day 120 for subjects receiving EPO on those days. The number and percent of subjects with a change in EPO regimen included dose increases and decreases; held doses were not included.

For quantitative baseline characteristics, the association with FGF-23 at Screening was evaluated with Pearson product moment and Spearman correlations. For qualitative baseline characteristics, the association with FGF-23 at Screening was evaluated with mean differences in FGF-23 among categories of the baseline characteristic.

Descriptive statistics for FGF-23 at Screening (mean, median, standard deviation, quartiles, minimum, and maximum) were summarized for subgroups.

Efficacy Results

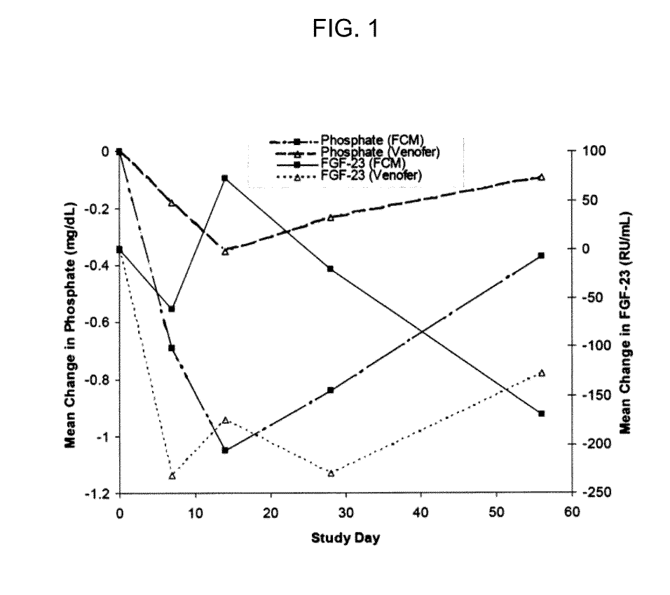

For the protocol-specified primary treatment group comparison, the mean increase in hemoglobin from baseline to the highest value between baseline and Day 56 or time of intervention demonstrated the noninferiority (i.e., lower limit of 2-sided 95% CI of treatment comparison was ≧−0.2) of FCM to Venofer (see e.g., FIG. 1). In addition, the mean increase was statistically significantly greater in the FCM group than in the Venofer group. When analyzed by baseline hemoglobin, use of EPO, and CKD stage, the mean increase in hemoglobin was numerically greater in the FCM group than that observed in the Venofer group for all subgroups and demonstrated noninferiority of FCM to Venofer for all comparisons. In addition, the mean increase was statistically significantly greater in the FCM group than in the Venofer group for baseline hemoglobin≧10.1 g/dL, no EPO use, use of EPO, and CKD stage 3-4.

Overall, the proportion of subjects with an increase in hemoglobin≧1.0 g/dL anytime between baseline and Day 56 or time of intervention demonstrated the noninferiority (i.e., lower limit of 2-sided 95% CI of treatment comparison was ≧7.5%) of FCM to Venofer. In addition, the proportion was statistically significantly greater in the FCM group than in the Venofer group. When analyzed by baseline hemoglobin, use of EPO, and CKD stage, the proportion of subjects with an increase in hemoglobin≧1.0 g/dL anytime between baseline and Day 56 or time of intervention achieved noninferiority for all comparisons except baseline hemoglobin≦9.0 g/dL and CKD stage 5. The proportion was statistically significantly greater in the FCM group than in the Venofer group for baseline hemoglobin≧10.1 g/dL, no EPO use, and CKD stage 3-4.

The mean increases in ferritin, TSAT, and serum iron and mean decreases in TIBC and unsaturated IBC from baseline to the highest value between baseline and Day 56 or time of intervention were statistically significantly greater in the FCM group than those observed in the Venofer group.

Safety Results

A total of 175 subjects (13.71%) in the FCM group and 156 subjects (12.14%) in the Venofer group had 1 or more adjudicated events comprising the primary composite safety endpoint. The 95% confidence interval for the treatment difference was −1.10% to 4.25%. This confidence interval includes zero and, therefore, provides evidence for equivalent cardiovascular risk in the 2 treatment groups. The most common component of the primary composite safety endpoint was protocol-defined hypertensive events in both the FCM (7.45%) and Venofer (4.36%) groups. A total of 70 subjects (5.49%) in the FCM group and 69 subjects (5.37%) in the Venofer group had 1 or more adjudicated events comprising the primary composite safety endpoint excluding protocol-defined hypertensive or hypotensive events. A total of 24 subjects (1.88%) in the FCM group and 35 subjects (2.72%) in the Venofer group had adjudicated events of death due to any cause, nonfatal myocardial infarction, or nonfatal stroke.

The hazard ratio (FCM versus Venofer) for the primary composite safety endpoint was 1.143 (CI=0.92, 1.42) based on Cox proportional hazards. The time-to-event curves differed between treatment groups during the first 14 days but were similar after Day 14 through Day 120. Since protocol-defined hypertensive and hypotensive events were more likely to be observed on dosing days (Days 0-14) when vital signs were more intensively monitored, the hazard ratio was also estimated after excluding protocol-defined hypertensive and hypotensive events. The re-estimated time-to-event curves were essentially the same for the 2 treatment groups (hazard ratio 1.017; CI=0.73, 1.42). The hazard ratio was also estimated for death due to any cause, nonfatal myocardial infarction, or nonfatal stroke. These re-estimated time-to-event curves trended nonsignificantly toward favoring FCM (hazard ratio 0.685; CI=0.41, 1.15). Since these 3 95% confidence intervals all include 1, further evidence is provided for equivalent cardiovascular risk in the 2 treatment groups.

There were 10 subjects (10.5%) in the FCM group who experienced at least 1 event of the composite safety endpoint in addition to a protocol-defined hypertensive event. Of these 10 subjects, 6 experienced the protocol-defined hypertensive event on a dosing day. Their protocol-defined hypertensive events occurred at the end of injection, and blood pressure had returned toward the pre-injection value at 30 minutes post-injection. The other events of the composite safety endpoint were reported more than 14 days after the hypertensive event. This supports the conclusion that these transient increases in blood pressure did not lead to other clinically significant sequelae.

There were 10 subjects (10.5%) in the FCM group who discontinued the study or study drug subsequent to a protocol-defined hypertensive event. Of these 10 subjects, 9 experienced the protocol-defined hypertensive event on a dosing day. One protocol-defined hypertensive event occurred pre-injection on Day 7 (Subject 301222) and the other events occurred at the end of injection. Blood pressure returned toward the pre-injection value following the end of injection. The most frequently reported adverse event resulting in discontinuation of study drug for these subjects was hypertension.

Most protocol-defined hypertensive events occurred on dosing days, and the blood pressure increases were transient. Little or no fluctuation in systolic or diastolic blood pressure was observed over the 120-day dosing period, except for the transient increases on dosing days. At baseline, the mean systolic and diastolic pressures and the percent of subjects with baseline systolic blood pressure>160 mmHg were higher in both treatment groups for subjects with protocol-defined hypertensive events as compared to subjects without protocol-defined hypertensive events. Additionally, systolic blood pressure was more variable for subjects with protocol-defined hypertensive events as compared to subjects without protocol-defined hypertensive events. Thus, the number of subjects who had 1 or more adjudicated events comprising the primary composite safety endpoint was similar in the FCM and Venofer groups, with the exception of subjects with protocol-defined hypertensive events. However, these events mainly occurred immediately after administration of study drug, were transient and were not associated with a persistent change in blood pressure or other clinically significant sequelae.

During the study, at least 1 treatment emergent adverse event was experienced by 65.4% (834/1276) of the subjects in the FCM group and 59.7% (767/1285) of the subjects in the Venofer group; this difference was statistically significant. The most commonly (˜5.0%) experienced treatment emergent adverse events in the FCM group were nausea (13.5%) and hypertension (10.7%). The most commonly experienced treatment emergent adverse event in the Venofer group was hypertension (7.5%).

During the study, at least 1 drug related treatment emergent adverse event (defined as possibly or probably related) was experienced by 23.4% (298/1276) of the subjects in the FCM group and 15.7% (202/1285) of the subjects in the Venofer group. Drug related treatment emergent adverse events experienced by ≧2.0% of subjects in the FCM group were nausea (110/1276; 8.6%), hypertension (59/1276; 4.6%), flushing (38/1276; 3.0%), dizziness (31/1276; 2.4%), and vomiting (26/1276; 2.0%). Drug related treatment emergent adverse events experienced by ≧2.0% of subjects in the Venofer group were dysgeusia (31/1285; 2.4%) and hypertension (26/1285; 2.0%). Most of these possibly or probably related events were severity Grade 1 or 2.

The majority of the treatment emergent adverse events experienced during the study (related or unrelated) were classified by the Investigator as Grade 1 or Grade 2 severity. Grade 3 treatment emergent adverse events were experienced by 165 subjects (12.9%) in the FCM group and 157 subjects (12.2%) in the Venofer group. Sixty (60) subjects (4.7%) in the FCM group and 59 subjects (4.6%) in the Venofer group experienced treatment emergent adverse events classified by the Investigator as Grade 4 severity. Fifteen (15) subjects (1.2%) in the FCM group and 18 subjects (1.4%) in the Venofer group experienced treatment emergent adverse events classified by the Investigator as Grade 5 (i.e., fatal events) severity.

During the study, at least 1 serious adverse event was experienced by 15.8% (202/1276) of the subjects in the FCM group and 15.3% (197/1285) of the subjects in the Venofer group. Cardiac failure congestive was the only serious adverse event experienced by ≧2.0% of subjects in either the FCM (30/1276; 2.4%) or Venofer (29/1285; 2.3%) group.

During the study, 33 (2.6%) subjects in the FCM group and 25 (1.9%) subjects in the Venofer group were prematurely discontinued from study drug due to the occurrence of adverse events. The majority of the adverse events resulting in premature discontinuation of study drug were considered possibly or probably related to study drug. At least 1 treatment emergent adverse event that resulted in discontinuation from the study was experienced by 31 (2.4%) subjects in the FCM group and 30 (2.3%) subjects in the Venofer group. The majority of these subjects had adverse events resulting in premature discontinuation from the study that were considered not related to study drug.

Screening FGF 23 was statistically significantly correlated with baseline hemoglobin, ferritin, TSAT, and GFR MDRD. High screening FGF 23 values were associated with low baseline values of hemoglobin, ferritin, TSAT, and GFR MDRD. A statistically significant decrease from baseline to Day 56 was observed within both the FCM and Venofer groups in FGF 23. A statistically significant difference was also observed within the Venofer group for the change from baseline to Day 7, Day 14, and Day 28.

Evaluations of physical examinations showed no clinically important differences between subjects in either the FCM or Venofer group. The only notable difference between the FCM and Venofer groups was for the proportion of subjects with PCS low phosphorus, which was greater in the FCM group (18.5%) compared with the Venofer group (0.8%). The change in phosphorus has been observed in other FCM clinical trials. But no adverse events associated with symptoms of low phosphate have been reported and no subject has discontinued therapy secondary to the low phosphate levels.

Conclusions

This study was conducted to assess the cardiovascular safety of FCM (2 injections of 750 mg) with that of an approved comparator regimen (Venofer; 5 injections of 200 mg). In this well-matched population, clinically important events were similar for both groups with a trend in favor of FCM for death, nonfatal myocardial infarction, and nonfatal stroke. In addition, FCM was well tolerated. Measures of efficacy were non-inferior or significantly better for the FCM dosing regimen used in this study.

### Example 3

### Iron Deficiency Anemia (IDA) and FGF23

The following example describes an open-label, multicenter, 5-week, prospective, randomized trial that compared single equivalent doses of intravenous elemental iron in the form of FCM versus iron dextran in women with iron deficiency anemia due to heavy uterine bleeding. Study visits were on Days 0 (when iron was infused), 1 (24 hours after iron infusion), 7, 14, and 35. The primary objectives were to determine the effect of iron deficiency on cFGF23 and iFGF23 levels and the effect of intravenous iron on iFGF23 and cFGF23 levels.

Subjects

Subjects were eligible to participate if they were age 18 years or older and met the following laboratory criteria at screening: hemoglobin<12 g/dL and serum ferritin≦100 ng/mL or ferritin≦300 ng/mL in combination with a transferrin saturation (TSAT)≦30%. Exclusion criteria included hypersensitivity to any component of FCM or iron dextran; serum phosphate<2.6 mg/dl at screening; history of hemochromatosis, untreated primary hyperparathyroidism, gastrointestinal malabsorption, malignancy within the previous 5 years, chronic kidney disease, end-stage renal disease, or kidney transplantation; treatment with intravenous iron within the previous 10 days; treatment with erythropoiesis stimulating agents, red blood cell transfusion, radiotherapy, chemotherapy, or surgical procedures requiring general anesthesia within the previous 30 days; AST or ALT levels greater than 1.5 times normal; pregnancy; and active alcohol or drug abuse. The study was approved by a central Institutional Review Board (Integreview, Austin, Tex.), and registered at Clinical Trials.gov (#NCT01307007). All subjects provided written informed consent.

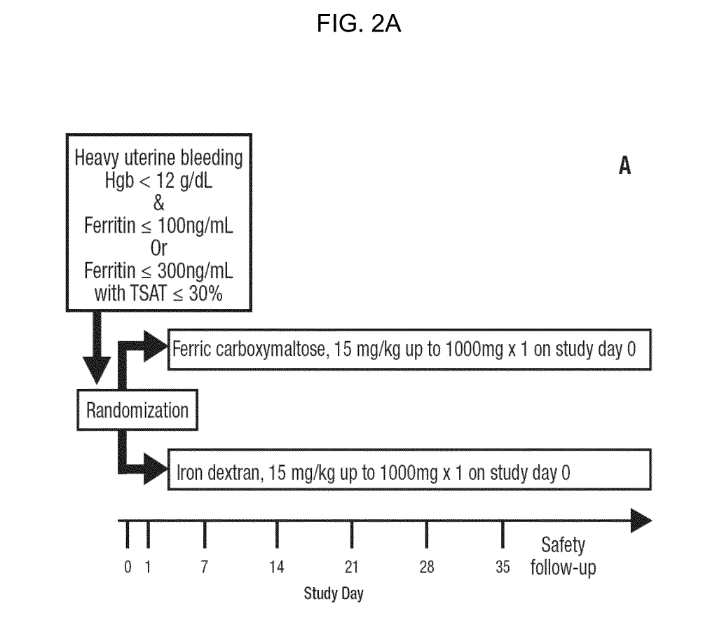

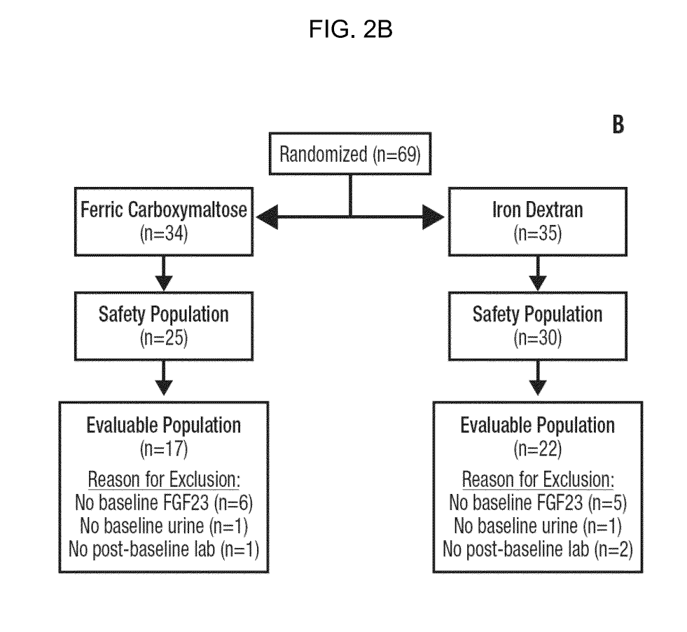

Sixty-nine subjects were randomized at four participating clinical centers, 34 to the FCM group and 35 to the iron dextran group (see e.g., FIG. 2B). Of these sixty-nine subjects, 25 subjects in the FCM group and 30 in the iron dextran group who received study drug were included in the safety population (the four centers contributed 12, 3, 10, and 30 participants, respectively). The evaluable population for the phosphate homeostasis analyses included 17 subjects in the FCM group and 22 in the iron dextran group (11, 2, 8, and 18 from the four centers, respectively).

Study Design

The design of the study and flow of subjects are presented in FIG. 2A and FIG. 2B. After informed consent was obtained, subjects who were found to be eligible entered the treatment phase of the study. Randomization occurred centrally via a fax-based system. Subjects who were randomized to FCM (Luitpold Pharmaceuticals Inc., Shirley N.Y.) received an intravenous infusion of 15 mg/kg up to a maximum of 1000 mg diluted in 250 cc normal saline solution. The complete dose was infused over 15 minutes on day 0. Subjects who were randomized to iron dextran received Dexferrum® (Luitpold Pharmaceuticals Inc., Shirley N.Y.) on day 0 using a standard protocol. An initial test dose of 25 mg was administered slowly over 5 minutes. After a 1-hour period of clinical observation, subjects who demonstrated no adverse reaction received the remainder of the dose, up to 15 mg/kg or 1000 mg including the test dose.

Laboratory testing of blood and three-hour urine collections was performed at the screening visit and on study days 0 (when iron was infused), 1 (24 hours after iron infusion), 7, 14, and 35 (see e.g., FIG. 2A). Any subject who experienced a reduction in serum phosphate below the normal range after day 0 underwent additional phosphate measurements at 14 day intervals following day 35 until the level returned to the normal reference range. Safety evaluations of all subjects included ascertainment of adverse events, physical examinations at all study visits, and laboratory assessments. Any subject who withdrew from the study received a follow-up phone call to ascertain adverse events 30 days after they received study drug.

Endpoints

The pre-specified primary endpoints were changes in blood and urine markers of phosphate and bone metabolism among the evaluable population, defined as those subjects with baseline and at least one post-randomization set of FGF23 and blood and urinary phosphate levels. Secondary endpoints included the proportion of subjects achieving a hemoglobin increase g/dL anytime between baseline and day 35, and the change from baseline to highest hemoglobin, ferritin, and TSAT anytime during the 35-day study period. Additional safety endpoints included incidence, severity, and seriousness of adverse events, and incidence of treatment-emergent abnormal clinical laboratory values in the safety population, defined as all subjects who received the study drug regardless of subsequent laboratory testing.

Laboratory Testing

Blood and urine samples were sent to a central laboratory for analysis (Covance Central Laboratory Service, Indianapolis, Ind.). Hemoglobin, blood counts, iron indices, electrolytes, and serum and urinary phosphate and creatinine were measured with standard, automated, multi-analyte techniques. Urinary fractional excretion of phosphate (FEPi) was calculated from the three-hour, morning urine collections as urinary phosphate x serum creatinine/serum phosphate x urinary creatinine. Serum levels of 25-hydroxyvitamin D and 1,25-dihydroxyvitamin D were measured in duplicate by radioimmunoassay (DiaSorin, Stillwater, Minn.) with intra-assay coefficients of variation (CV)<11.6%. Plasma C-terminal FGF23 (cFGF23) was measured in duplicate in EDTA-plasma samples using a human FGF23 immunometric assay capable of detecting both the intact peptide and its C-terminal fragments (Immutopics, San Clemente, Ca) with intra-assay CV≦3.8%. Because cFGF23 testing was performed in parallel with rolling enrollment and follow-up, all cFGF23 assay plates tested combinations of baseline and post-intervention samples from multiple study participants. Serum intact FGF23 (iFGF23) was measured in batches of baseline and post-intervention samples from multiple study participants at study end using an immunometric assay that detects the intact peptide exclusively (Kyowa Medex Company, Shizuoka, Japan) with intra-assay CV<2.7%. Serum hepcidin levels were measured on days 0, 7, and 35 using an enzyme-linked immunosorbent assay (Intrinsic LifeSciences, La Jolla, Calif.) with intra-assay CV≦19%. Urinary amino acids, glucose, albumin, and beta 2 microglobulin were measured to test for the possibility of global proximal tubular dysfunction. Urinary deoxypyridinoline cross link, and serum osteocalcin and bone-specific alkaline phosphatase were measured to assess bone turnover.

Statistical Methods

It was estimated that a sample size of 20 subjects per arm would provide >90% power to detect a within-group change in serum phosphate from baseline of at least 0.8 times the standard deviation of mean baseline phosphate. This sample would provide >90% power to detect a between-group difference in change from baseline in serum phosphate of at least 1.1 times the standard deviation of the mean change.

Baseline characteristics are presented as mean±standard error for continuous variables and as proportions for categorical variables. The association between iron indices and baseline and change in FGF23 levels was assessed with Spearman correlations. Restricted maximum likelihood-based repeated measures analyses (linear mixed models) that considered the effects of treatment, visit, and treatment x time interaction, with baseline values treated as a fixed covariate were used for the primary analyses of mean changes from baseline in continuous markers of phosphate homeostasis. The models employed unstructured covariance structures without imputing missing values. All outcome variables that were not normally distributed were analyzed after natural log-transformation. Changes from baseline to each scheduled evaluation and to the minimum, maximum, and final value within treatment groups were compared to zero with Wilcoxon signed-ranks tests, and between-group differences were compared with Wilcoxon rank sum tests.

The 2-sided, 95% confidence interval for the absolute difference in the proportions between the FCM and iron dextran groups was calculated to test the secondary endpoint of whether the proportion of subjects who achieved a hemoglobin increase ≧2 g/dL at any time after the baseline visit differed by iron preparation. This was an intention-to-treat analysis such that any subjects who withdrew from the study prior to having a post-baseline hemoglobin assessment were considered to have failed to achieve a ≧2 g/dL increase. Paired t-tests were used to analyze whether the intravenous iron formulations significantly increased continuous measures of hemoglobin, TSAT, ferritin, and hepcidin from baseline to the highest value among evaluable subjects within each treatment group. The number and proportion of subjects who experienced an adverse event in the safety population was summarized and rates between the iron treatment groups using Fisher's exact test were compared for the categorical safety endpoints. Analyses were conducted using SAS version 9 (Cary, N.C.). All statistical tests were 2-tailed and p values<0.05 were considered statistically significant.

Baseline Assessment

Baseline characteristics were similar between the groups in the evaluable population except for a greater proportion of African Americans in the FCM arm (see e.g., TABLE 3). Serum phosphate, FEPi, and iFGF23 levels were normal, but cFGF23 levels were markedly elevated in both groups (overall mean 807.8±123.9 RU/ml). Baseline cFGF23 levels were negatively correlated with baseline iron levels of (r=−0.61; p<0.001), TSAT (r=−0.67; P<0.01), ferritin (r=−0.55; p<0.001), and hepcidin (r=−0.32; P=0.049). In contrast, baseline iFGF23 levels correlated positively and less strongly with iron levels (r=0.41; p=0.008) and TSAT (r=0.39; p=0.01), and did not correlate with ferritin (r=0.21; p=0.19) or hepcidin levels (r=0.21; p=0.21).

TABLE 3
Demographic and baseline clinical characteristics of the
evaluable population. Continuous variables are presented as mean ±
standard error, and categorical variables as proportions. The
evaluable population included subjects with baseline and at least
one post-randomization set of FGF23 and blood and urinary
phosphate levels.

| | Ferric | |
|---|---|---|
| | carboxymaltose | Iron dextran |
| | n = 17 | n = 22 |
| Demographics | | |
| Age, years | 35.5 ± 2.5 | 34.3 ± 2.3 |
| Race, n (%) | | |
| African American | 14 (82) | 11 (50) |
| Caucasian | 2 (12) | 8 (36) |
| Hispanic | 1 (6) | 3 (14) |
| Body mass index, kg/m² | 32.0 ± 1.9 | 33.6 ± 2.0 |
| Iron deficiency parameters | | |
| Hemoglobin, g/dL | 9.5 ± 0.3 | 9.8 ± 0.3 |
| Iron, μg/dL | 24.2 ± 4.2 | 34.0 ± 4.2 |
| Transferrin saturation, % | 6.5 ± 1.2 | 9.9 ± 2.4 |
| Ferritin, ng/mL | 4.4 ± 0.6 | 6.9 ± 1.7 |
| Hepcidin, ng/mL | 14.3 ± 0.5 | 16.7 ± 0.6 |
| Previous iron therapy, n (%) | 6 (35) | 11 (50) |
| Mineral metabolism parameters | | |
| Serum phosphate, mg/dL | 3.3 ± 0.1 | 3.5 ± 0.1 |
| Fractional excretion of phosphate, % | 11.9 ± 0.9 | 11.7 ± 0.9 |
| Serum calcium, mg/dL | 9.4 ± 0.1 | 9.4 ± 0.1 |
| Serum parathyroid hormone, | 56.2 ± 4.9 | 57.5 ± 5.4 |
| pg/mL | | |
| Serum 25(OH)vitamin D, ng/mL | 16.0 ± 1.2 | 17.7 ± 1.6 |
| Serum 1,25(OH)₂vitamin D, pg/mL | 50.7 ± 3.2 | 49.5 ± 2.5 |
| Serum intact FGF23, pg/mL | 28.6 ± 1.4 | 28.4 ± 1.6 |
| Plasma C-terminal FGF23, | 783 ± 168 | 827 ± 181 |
| RU/mL | | |

Effects of Iron Treatment on Anemia and Iron Indices

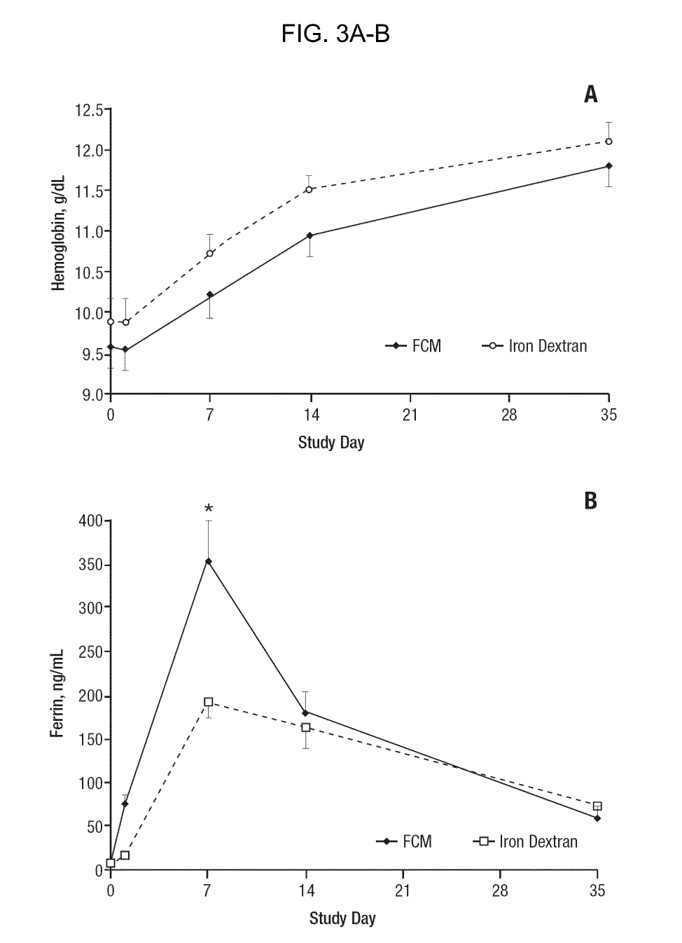

The mean total doses of elemental iron were 918 mg in the FCM group and 911 mg in the iron dextran group. Hemoglobin increased by 2.0 g/dL in 60% of the FCM group and 53% of the iron dextran group (absolute difference 6.7%; 95% confidence interval [CI] −23.2, 36.6%; p=0.79), with no difference in the temporal change between groups (see e.g., FIG. 3A, TABLE 3). FCM induced a greater increase in serum ferritin by day 7 compared to iron dextran, but thereafter, the levels were similar (see e.g., FIG. 3B, TABLE 3). Both FCM and iron dextran increased TSAT and serum hepcidin levels significantly (p≦0.001) (see e.g., TABLE 3). By day 7, both FCM and iron dextran significantly increased serum hepcidin levels, which remained significantly elevated compared to baseline in both groups on day 35 (data not shown).

TABLE 3
Effects of iron therapies on erythropoiesis and iron
parameters.

| | Ferric | Iron | |
|---|---|---|---|
| | carboxymaltose | dextran | |
| | n = 25 | n = 30 | P^{†} |
| Hemoglobin increase ≧2.0 g/dL, | 15 (60) | 16 (53) | 0.79 |
| n (%)* | | | |
| Mean increase in hemoglobin from | 2.0 ± 0.3 | 2.2 ± 0.2 | 0.27 |
| baseline to highest value, g/dL | | | |
| Mean increase in ferritin from | 306 ± 41 | 189 ± 21 | 0.02 |
| baseline to highest value, ng/dL | | | |
| Mean increase in transferrin | 68 ± 6.4 | 44 ± 3.0 | 0.002 |
| saturation from baseline to highest | | | |
| value, % | | | |
| Mean increase in hepcidin from | 33.4 | 77.8 | 0.04 |
| baseline to highest value, ng/mL | | | |

Continuous variables are presented as mean ± standard error, and categorical variables as proportions.
^{†}P for between group differences.
*This analysis was intention-to-treat in the safety population, which included all subjects who received study drug regardless of whether they completed subsequent laboratory testing.

Effects of Iron Treatment on Serum and Urinary Phosphate

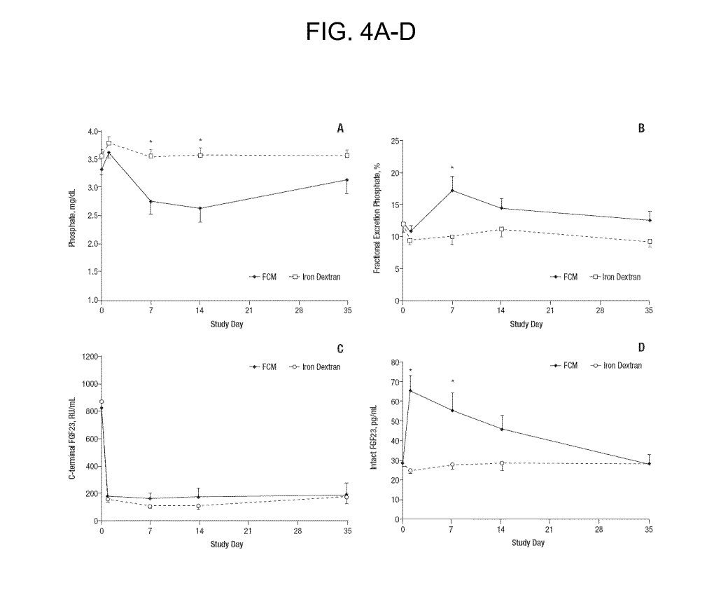

In both the FCM and iron dextran groups, serum phosphate increased modestly within 24 hours after iron administration (see e.g., FIG. 4A). In the iron dextran group, serum phosphate returned to near baseline levels by day 7 and remained stable for the remainder of the study. In the FCM group, serum phosphate decreased significantly by 0.6 mg/dL by day 7, and by 0.7 mg/dL by day 14, before returning towards normal by day 35 (see e.g., FIG. 4A). Serum phosphate decreased to <2.0 mg/dL in 10 subjects in the FCM group and none in the iron dextran group. Among the 6 subjects whose serum phosphate remained below the normal range at day 35, levels normalized in all by day 80.

The mean urinary fractional excretion of phosphate (FEPi) trended downwards on day 1 in both the FCM and iron dextran groups, but by day 7, mean urinary FEPi increased significantly compared with baseline in the FCM group (p=0.025), and remained elevated through day 35 (see e.g., FIG. 4B).

There was no significant change over time in FEPi in the iron dextran group. There was no evidence of glycosuria, amino aciduria, or albuminuria in either group to suggest generalized proximal tubular dysfunction contributed to reduced serum phosphate, and there were no significant differences in bone turnover markers between the groups (data not shown).

Effects of Iron Treatment on cFGF23 and iFGF23 Levels

In the overall study population, cFGF23 decreased by 81.4%±14.9% by day 1, and remained unchanged throughout the remainder of the study period without significant differences between the FCM and iron dextran groups (see e.g., FIG. 4C, TABLE 4). The magnitude of reduction in cFGF23 from baseline to minimum value within individual subjects was greatest among those with the most severe iron deficiency at baseline (correlation of baseline ferritin with maximal change in cFGF23, r=0.55; p<0.001).

Although there were no significant changes in iFGF23 levels in response to iron dextran, iFGF23 levels increased significantly by day 1 in the FCM group, remained elevated through days 7 and 14, before returning to baseline by day 35 (see e.g., FIG. 4D, TABLE 4). The increase in iFGF23 from day 0 to 1 in the FCM group correlated significantly with the concomitant increase in ferritin (r=0.59; p<0.001) and the magnitude of the subsequent decrease in serum phosphate at day 7 (r=−0.50; p=0.002).

TABLE 4
C-terminal and intact FGF23 levels throughout the study
period.

| | C-terminal FGF23 | | Intact FGF23 | |
|---|---|---|---|---|
| | | Iron | | Iron |
| | FCM | dextran | FCM | dextran |
| Baseline | 783 ± 168 | 827 ± 181 | 28.6 ± 1.4 | 28.4 ± 1.6 |
| Day 1 | 177 ± 12.4 | 152 ± 17.5 | 65.2 ± 8.0 | 24.6 ± 1.9 |
| Change from | −78.6 | −82.5 | 128.0 | −13.4 |
| baseline, % | | | | |
| Day 7 | 165 ± 34.9 | 114 ± 17.7 | 54.9 ± 18.0 | 27.5 ± 1.7 |
| Change from | −80.9 | −85.6 | 91.8 | −4.4 |
| baseline, % | | | | |
| Day 14 | 170 ± 55.8 | 107 ± 16.0 | 45.8 ± 6.9 | 28.4 ± 2.1 |
| Change from | −77.5 | −87.3 | 59.1 | −1.3 |
| baseline, % | | | | |
| Day 35 | 182 ± 73.7 | 170 ± 51.2 | 28.1 ± 2.9 | 28.3 ± 1.7 |
| Change from | −78.9 | −80.0 | 2.0 | −9.3 |
| baseline, % | | | | |

Results are presented as mean ± standard error.
FCM is Ferric carboxymaltose.

Effects of Iron Treatment on Vitamin D, Calcium and PTH Levels

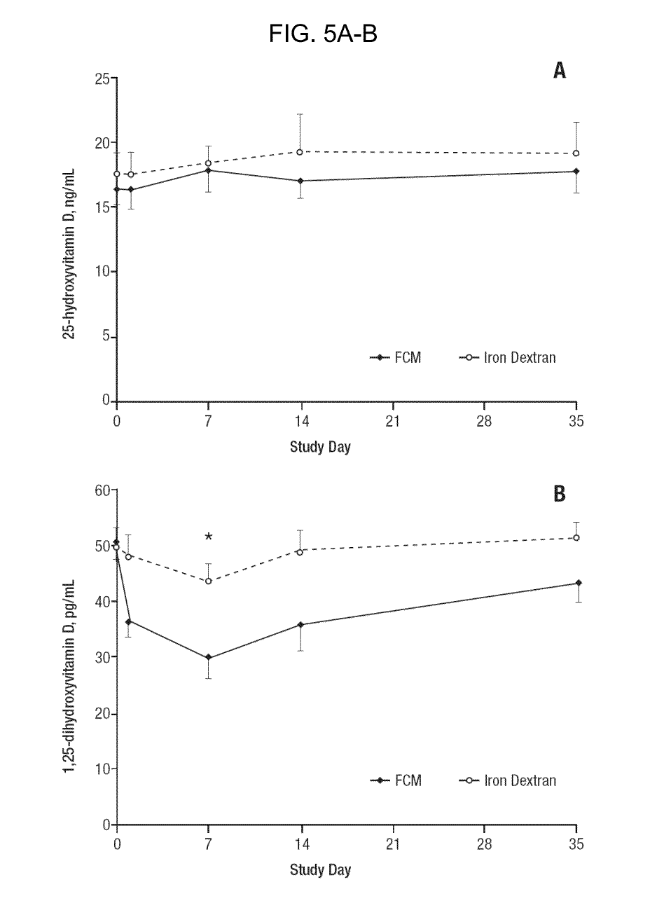

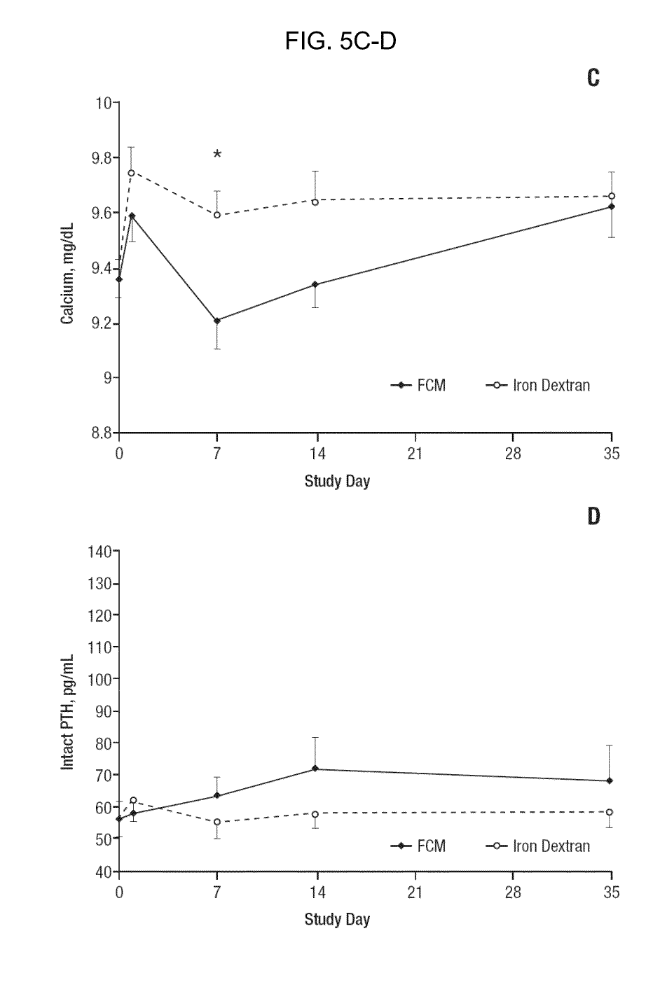

Neither FCM nor iron dextran induced a significant change in 25-hydroxyvitamin D levels (see e.g., FIG. 5A. Iron dextran did not induce a significant change in 1,25-dihydroxyvitamin D levels. In the FCM group, 1,25-dihydroxyvitamin D levels fell significantly from baseline to day 1 and reached a nadir by day 7 before returning towards normal by day 35 (see e.g., FIG. 5B). In the iron dextran group, serum calcium increased within 24 hours, returned towards baseline by day 7 and remained stable for the remainder of the study (see e.g., FIG. 5C). In the FCM group, serum calcium increased within 24 hours, then decreased significantly below baseline on day 7 before returning towards normal by day 35 (see e.g., FIG. 5C). Although the changes in PTH levels were not significant in either group, there was a trend towards an increase in PTH in the FCM group that peaked at day 14 (p=0.10; see e.g., FIG. 5D).

Stratified Analyses by Development of Serum Phosphate<2.0 mg/dL

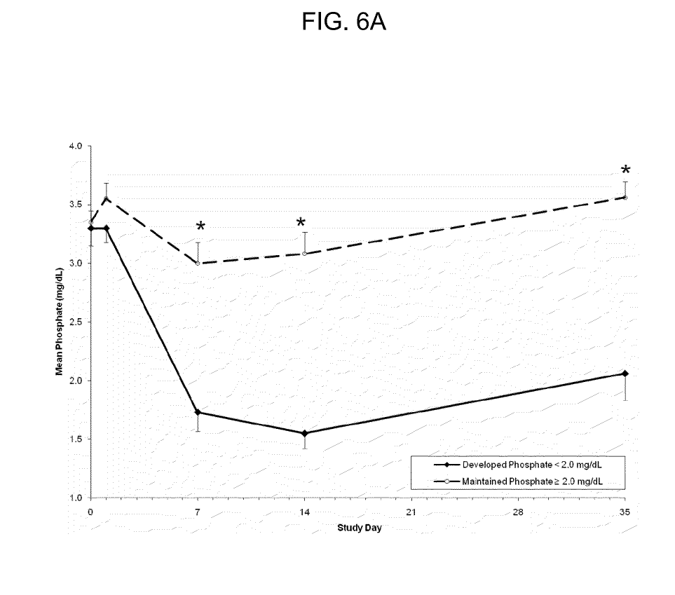

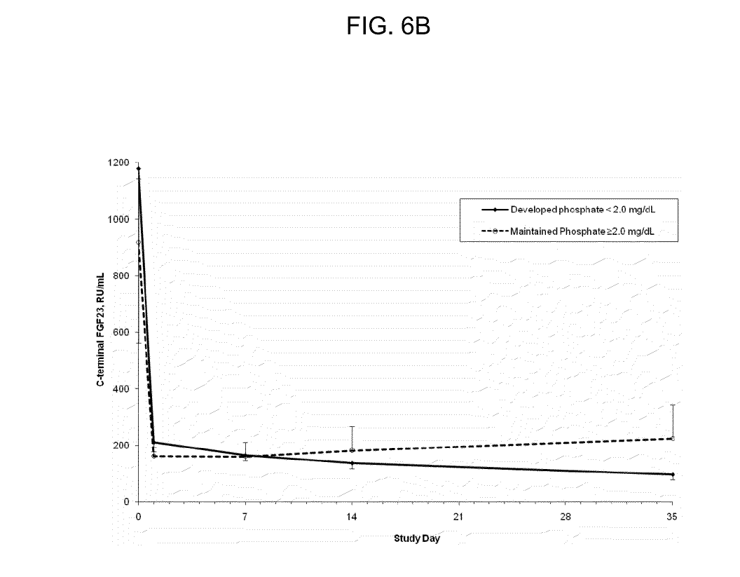

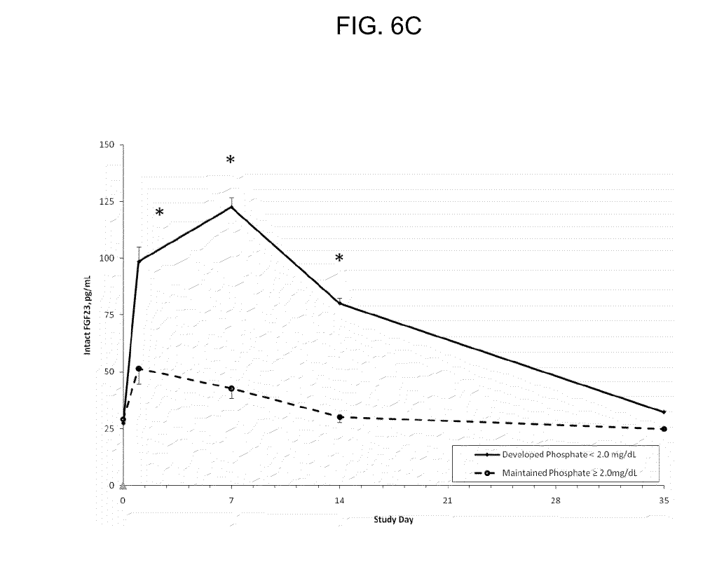

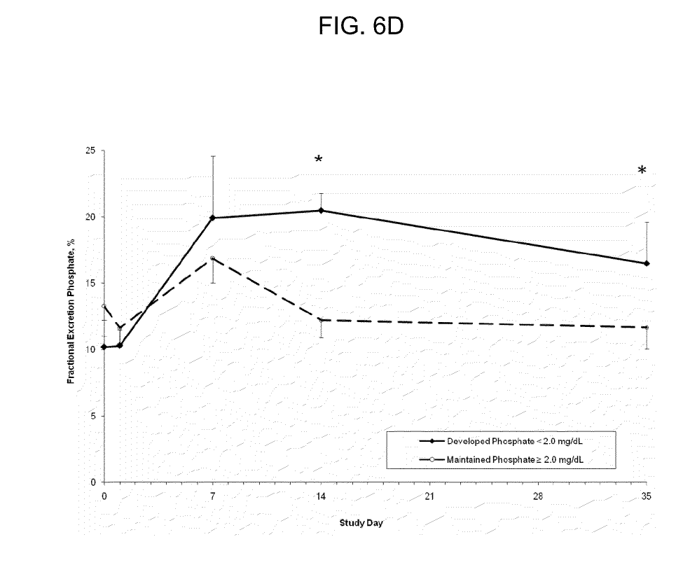

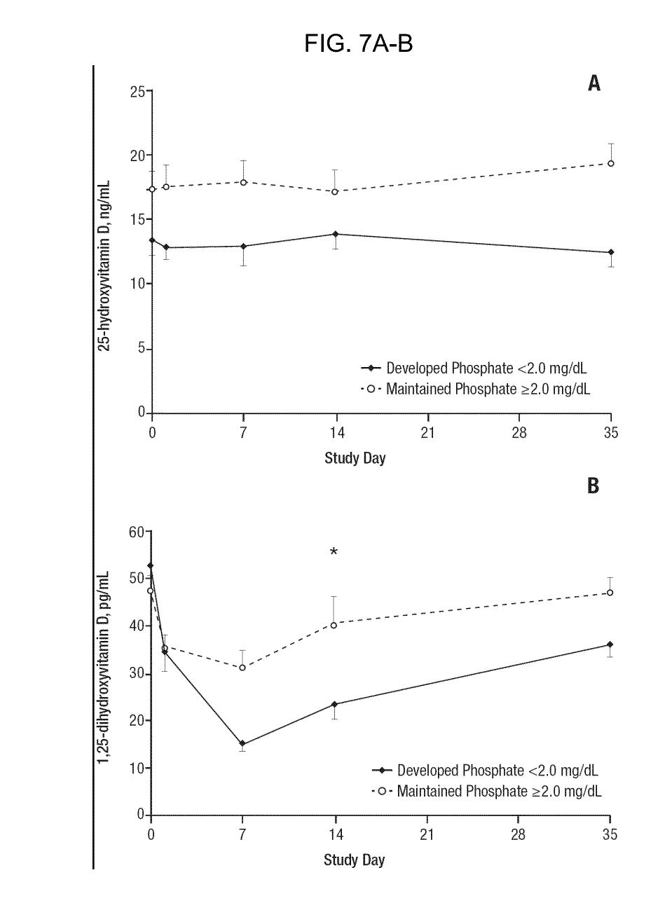

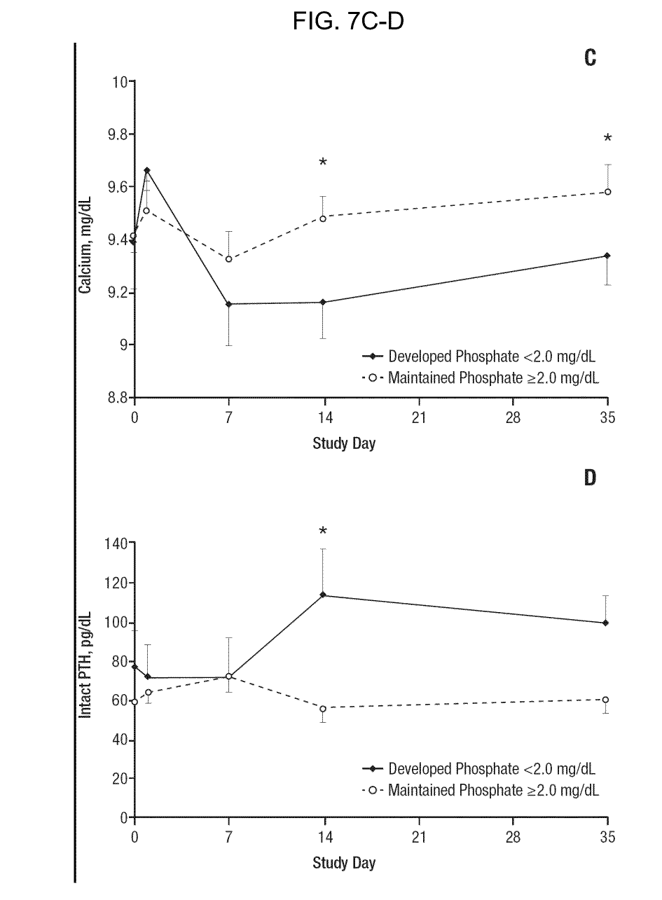

Laboratory results were compared from the 10 subjects who received FCM and developed serum phosphate levels<2.0 mg/dL with results from the remaining subjects who received FCM but maintained a serum phosphate 2.0 mg/dL to further characterize the effects of FCM on phosphate homeostasis. The nadir in serum phosphate occurred at day 14 (see e.g., FIG. 6A). Although cFGF23 decreased similarly in both groups (see e.g., FIG. 6B), those who developed serum phosphate<2.0 mg/dL manifested greater iFGF23 by day 1 that persisted through day 14 before normalizing by day 35 (see e.g., FIG. 6C). FEPi increased to a significantly greater extent in the low phosphate subgroup, peaked by day 14, and remained significantly elevated through day 35 (see e.g., FIG. 6D). Levels of 25-hydroxyvitamin D did not change over time between groups (FIG. 6A). In contrast, levels of 1,25-dihydroxyvitamin D and serum calcium decreased to a greater extent in the low phosphate subgroup, reaching a nadir by day 7 before returning towards normal (see e.g., FIG. 7B, FIG. 7C). Beginning at day 14, PTH levels were significantly higher in the low phosphate subgroup and decreased only partially by day 35 (see e.g., FIG. 7D).

Safety Results

In the FCM group, 17 subjects (68%) experienced one or more adverse events, 8 subjects (32%) of which were considered to be related to study drug (see e.g., TABLE 5). In the iron dextran group, 18 subjects (60%) experienced one or more adverse events, 11 subjects (37%) of which were considered to be related to study drug. There were no significant differences between the groups. Reduced serum phosphate noted by subjects' physicians was the most common adverse event reported in the FCM group (32%), and occurred significantly more frequently than in the iron dextran group (3%; p=0.008).

TABLE 5
Adverse events. Results are presented as proportions.

| | Ferric | | |
|---|---|---|---|
| | carboxymaltose | Iron Dextran | |
| | n = 25 | n = 30 | P |
| Patients experiencing one or more | 17 (68%) | 18 (60%) | 0.59 |
| adverse events, n (%) | | | |
| Patients experiencing one or more | 8 (32%) | 11 (37%) | 0.78 |
| adverse events related to study | | | |
| drug, n (%) | | | |
| Patients experiencing one or more | 0 (0%) | 0 (0%) | 1.00 |
| serious adverse events, n (%) | | | |
| Discontinuation from study due to | 2 (8%) | 2 (6%) | 1.00 |
| adverse event, n (%) | | | |

Conclusions

This study was conducted to assess the effects of IDA on FGF levels, the effects of iron treatment on anemia and iron indices, the effects of iron treatment on cFGF23 and iFGF23 levels, and the effects of iron treatment on 1,25-dihydroxyvitamin D, calcium, and PTH levels.

This randomized physiological study of intravenous iron therapy for iron deficiency anemia yields new insight into the relationship between iron and phosphate homeostasis. The first novel finding is that iron deficiency anemia is associated with normal iFGF23 but markedly elevated cFGF23 levels to an extent rarely seen except in renal failure or hereditary rachitic diseases (23). Second, rapid correction of iron deficiency with different intravenous iron preparations reduced cFGF23 levels by approximately 80% within 24 hours. This study provides the first direct evidence in humans that iron deficiency causes cFGF23 levels to rise, as was proposed in the seminal study of FGF23 in iron deficient mice (21).

The third novel finding was that FCM but not iron dextran induced a significant increase in iFGF23 levels by 24 hours, which was associated with a subsequent increase in FEPi, and decreases in serum phosphate, 1,25-dihydroxyvitamin D and calcium levels that were followed by an increase in PTH. This cascade was accentuated among subjects who developed the greatest reductions in serum phosphate. The latter results recapitulate previously reported findings of acute, FGF23-mediated phosphate wasting in response to intravenous iron (11-18). The results exclude the hypothesis that massive phosphate uptake by developing red blood cells lowered serum phosphate, because this would have caused urinary phosphate excretion to decrease. Low serum phosphate persisted in some subjects at day 35 when FEPi and PTH were persistently elevated but iFGF23 levels had already normalized. This supports that transient secondary hyperparathyroidism due to FGF23-mediated reduction in 1,25-dihydroxyvitamin D and calcium levels also contributes to reduced serum phosphate in response to intravenous iron. Thus, in this study, the previously reported paradoxical findings that iron deficiency induces fgf23 transcription was reproduced, whereas its correction using certain forms of high-dose iron therapy can lower serum phosphate by raising circulating iFGF23 levels despite seemingly reducing transcription as suggested by rapidly reducing cFGF23 levels. Collectively, these findings show a relationship between iron status, intravenous iron preparations and regulation of FGF23 synthesis and degradation.

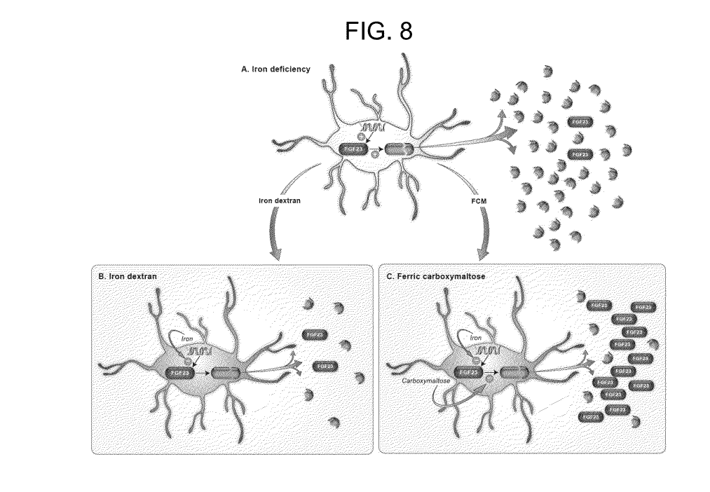

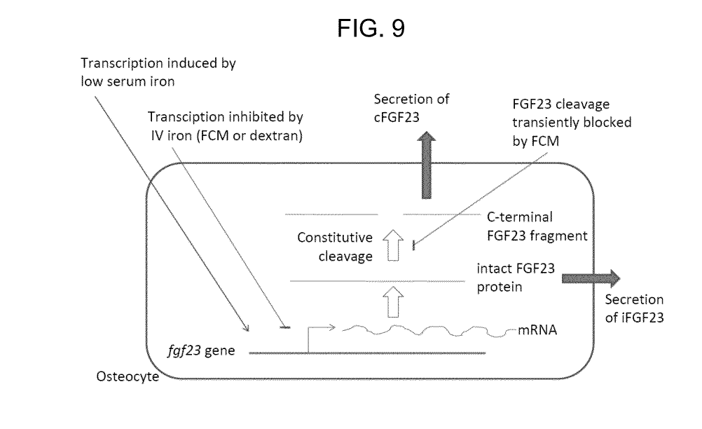

Iron deficiency stimulates fgf23 transcription in osteocytes, but does not cause hypophosphatemia in wild-type mice because of increased intracellular degradation of FGF23, which leaves a footprint of elevated circulating cFGF23 but normal iFGF23 levels (see e.g., FIG. 8A) (21;22). The finding that both FCM and iron dextran rapidly lowered cFGF23 validates these aspects of FGF23 regulation in humans and supports that the iron component these agents share in common likely drove the effect (see e.g., FIG. 8B, FIG. 8C). In contrast, the acute increase in iFGF23 following FCM but not iron dextran supports that the main difference between the agents, namely their carbohydrate moieties, likely affect an additional aspect of FGF23 regulation. There are several possible hypotheses to explain these results. First, iron dextran and FCM could both reduce fgf23 transcription, while FCM simultaneously inhibits FGF23 degradation in osteocytes (see e.g., FIG. 8B, FIG. 8C). This could be achieved by enhancing post-translational 0-glycosylation of FGF23, which protects it from cleavage (24) by inhibiting the currently unidentified system that degrades FGF23 in osteocytes, or they accelerating iFGF23 secretion before it can be degraded. Second, different iron preparations may differentially reduce peripheral degradation or clearance of circulating iFGF23 after it is secreted by the osteocyte. Third, certain iron preparations could induce ectopic production of FGF23 by other organs that are involved in iron metabolism and have the capacity to express FGF23, including the liver and lymphatic system (25).

Several participants who were initially randomized never returned to receive study drugs, and several more received study drugs but had incomplete laboratory testing. In the absence of complete laboratory data, these participants could not be included in the pre-specified primary analyses of biochemical changes in mineral metabolism. But since loss to follow-up appears to have been unrelated to randomized group, it is unlikely to have affected the primary results. This was a small open-label trial of short duration from which we cannot extrapolate conclusions about the long-term safety of FCM. However, previous studies demonstrated an equivalent safety profile of FCM relative to other commercially available iron preparations, aside from transient reductions in serum phosphate levels that occurred in up to 60% (2-10). A randomized trial of 2500 patients with chronic kidney disease and elevated cardiovascular risk that compared FCM to iron sucrose during 120 days of follow-up found no significant differences between groups in adverse events or the adjudicated composite end point of major cardiovascular events, despite a higher incidence of serum phosphate<2.0 mg/dL in the FCM arm (18.5% versus 0.8%)(26) (see e.g., Example 2). These studies support that reduced serum phosphate following FCM is not associated with adverse clinical outcomes. Although this trial was conducted exclusively in pre-menopausal women, many of whom were African American, previous studies did not report racial or gender differences in serum phosphate levels following FCM, there may be differences in bone and mineral metabolism between African-Americans and Caucasians that may include regulation of FGF23.

In summary, a single dose of both FCM and iron dextran was shown to significantly increase hemoglobin and iron indices in this randomized study of women with iron deficiency anemia due to heavy uterine bleeding. Although cFGF23 levels fell in both groups, an increase in iFGF23 was believed to mediate the transient and asymptomatic reductions in serum phosphate levels that were observed exclusively in the FCM-treated subjects.

1. A method of treating a disease or disorder associated with increased phosphorus or increased cFGF23 levels in a subject comprising:
administering an iron carbohydrate complex to a subject in need thereof;
wherein,
a phosphorus level of the subject decreases;
a fractional excretion of phosphate of the subject increases;
a C-terminal FGF23 (cFGF23) level of the subject decreases; or
an intact FGF23 (iFGF23) level of the subject increases.

2. (canceled)

3. The method of claim 1, wherein the or disorder associated with increased phosphorus or increased cFGF23 levels comprises chronic kidney disease, hypophosphotemia, or a cardiac disease or condition.

4. The method of claim 1, wherein
the iron carbohydrate complex comprises maltose;
the phosphorus level of the subject decreases;
the fractional excretion of phosphate of the subject increases;
the C-terminal FGF23 (cFGF23) level of the subject decreases; and
the intact FGF23 (iFGF23) level of the subject increases.

5. The method of claim 1, wherein the subject is diagnosed with (i) an elevated level of phosphorus, (ii) an elevated level of cFGF23, or (iii) an elevated level of phosphorus and an elevated level of cFGF23.

6. The method of claim 1, wherein the subject is diagnosed with chronic kidney disease, hypophosphotemia, or a cardiac disease or condition.

7. The method of claim 1, wherein the subject is diagnosed with a phosphorus level above a baseline.

8. The method of claim 1, wherein the subject is diagnosed with a cFGF23 level above a baseline.

9. The method of claim 3, wherein the cardiac disease or condition is selected from the group consisting of: chronic heart damage, chronic heart failure, cardiac damage resulting from injury or trauma, cardiac damage resulting from a cardiotoxin, cardiac damage from radiation or oxidative free radicals, cardiac damage resulting from decreased blood flow, and myocardial infarction.

10. The method of claim 9, wherein the cardiotoxin comprises cFGF23.

11. The method of claim 1, wherein
the phosphorus level of the subject decreases by at least about 10%, at least about 20%, at least about 30%, at least about 40%, at least about 50%, at least about 60%, at least about 70%, at least about 80%, at least about 90%, or at least about 100%; or
the fractional excretion of phosphate increases by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 40%, at least about 50%, at least about 60%, at least about 70%, at least about 80%, at least about 90%, or at least about 100%.

12. The method of claim 1, wherein the phosphorus level of the subject decreases by at least about 0.01 mg/dL, at least about 0.05 mg/dL, at least about 0.1 mg/dL, at least about 0.2 mg/dL, at least about 0.3 mg/dL, at least about 0.4 mg/dL, at least about 0.5 mg/dL, at least about 0.6 mg/dL, at least about 0.7 mg/dL, at least about 0.8 mg/dL, at least about 0.9 mg/dL, at least about 1.0 mg/dL, at least about 1.1 mg/dL, at least about 1.2 mg/dL, at least about 1.3 mg/dL, at least about 1.4 mg/dL, at least about 1.5 mg/dL, at least about 1.6 mg/dL, at least about 1.7 mg/dL, at least about 1.8 mg/dL, at least about 1.9 mg/dL, at least about 2.0 mg/dL, at least about 2.1 mg/dL, at least about 2.2 mg/dL, at least about 2.3 mg/dL, at least about 2.4 mg/dL, at least about 2.5 mg/dL, at least about 2.6 mg/dL, at least about 2.7 mg/dL, at least about 2.8 mg/dL, at least about 2.9 mg/dL, at least about 3.0 mg/dL, at least about 3.1 mg/dL, at least about 3.2 mg/dL, at least about 3.3 mg/dL, at least about 3.4 mg/dL, or at least about 3.5 mg/dL.

13. The method of claim 1, wherein the cFGF23 level of the subject decreases by at least about 10%, at least about 20%, at least about 30%, at least about 40%, at least about 50%, at least about 60%, at least about 70%, at least about 80%, at least about 90%, or at least about 100%.

14. The method of claim 1, wherein the cFGF23 level of the subject decreases by at least about 50 RU/mL, at least about 100 RU/mL, at least about 150 RU/mL, at least about 200 RU/mL, at least about 250 RU/mL, at least about 300 RU/mL, at least about 350 RU/mL, at least about 400 RU/mL, at least about 450 RU/mL, at least about 500 RU/mL, at least about 550 RU/mL, at least about 600 RU/mL, at least about 650 RU/mL, at least about 700 RU/mL, at least about 750 RU/mL, at least about 800 RU/mL, at least about 850 RU/mL, at least about 900 RU/mL, at least about 1,000 RU/mL, at least about 1,100 RU/mL, at least about 1,200 RU/mL, at least about 1,300 RU/mL, at least about 1,400 RU/mL, at least about 1,500 RU/mL, at least about 1,600 RU/mL, at least about 1,700 RU/mL, at least about 1,800 RU/mL, at least about 1,900 RU/mL, or at least about 2,000 RU/mL.

15. The method of claim 1, wherein the iFGF23 level of the subject increases by at least about 10%, at least about 20%, at least about 30%, at least about 40%, at least about 50%, at least about 60%, at least about 70%, at least about 80%, at least about 90%, or at least about 100%.

16. The method of claim 1, wherein
the iFGF23 level of the subject increases by at least about 10 RU/mL, at least about 20 RU/mL, at least about 30 RU/mL, at least about 40 RU/mL, at least about 50 RU/mL, at least about 60 RU/mL, at least about 70 RU/mL, at least about 80 RU/mL, at least about 90 RU/mL, at least about 100 RU/mL, at least about 110 RU/mL, at least about 120 RU/mL, at least about 130 RU/mL, at least about 140 RU/mL, at least about 150 RU/mL, at least about 200 RU/mL, at least about 250 RU/mL, at least about 300 RU/mL, at least about 350 RU/mL, at least about 400 RU/mL, at least about 450 RU/mL, at least about 500 RU/mL, at least about 550 RU/mL, at least about 600 RU/mL, at least about 650 RU/mL, at least about 700 RU/mL, at least about 750 RU/mL, at least about 800 RU/mL, at least about 850 RU/mL, at least about 900 RU/mL, or at least about 1,000 RU/mL; or
the iFGF23 level of the subject increases by at least about 10 pg/mL, at least about 20 pg/mL, at least about 30 pg/mL, at least about 40 pg/mL, at least about 50 pg/mL, at least about 60 pg/mL, at least about 70 pg/mL, at least about 80 pg/mL, at least about 90 pg/mL, at least about 100 pg/mL, at least about 110 pg/mL, at least about 120 pg/mL, at least about 130 pg/mL, at least about 140 pg/mL, at least about 150 pg/mL, at least about 160 pg/mL, at least about 170 pg/mL, at least about 180 pg/mL, at least about 190 pg/mL, at least about 200 pg/mL, at least about 250 pg/mL, at least about 300 pg/mL, at least about 350 pg/mL, at least about 400 pg/mL, at least about 450 pg/mL, at least about 500 pg/mL, at least about 550 pg/mL, at least about 600 pg/mL, at least about 650 pg/mL, at least about 700 pg/mL, at least about 750 pg/mL, at least about 800 pg/mL, at least about 850 pg/mL, at least about 900 pg/mL, or at least about 1,000 pg/mL.

17. The method of claim 1, wherein the iron carbohydrate complex comprises one of: an iron carboxymaltose or an iron dextran.

18. The method of claim 1, wherein the iron carbohydrate complex comprises maltose.

19. The method of claim 1, wherein
the iron carbohydrate complex comprises an iron carboxymaltose;
the phosphorus level of the subject decreases by at least about 0.01 mg/dL, at least about 0.05 mg/dL, at least about 0.1 mg/dL, at least about 0.2 mg/dL, at least about 0.3 mg/dL, at least about 0.4 mg/dL, at least about 0.5 mg/dL, at least about 0.6 mg/dL, at least about 0.7 mg/dL, at least about 0.8 mg/dL, at least about 0.9 mg/dL, at least about 1.0 mg/dL, at least about 1.1 mg/dL, at least about 1.2 mg/dL, at least about 1.3 mg/dL, at least about 1.4 mg/dL, or at least about 1.5 mg/dL;
the cFGF23 level of the subject decreases by at least about 100 RU/mL, at least about 150 RU/mL, at least about 200 RU/mL, at least about 250 RU/mL, at least about 300 RU/mL, at least about 350 RU/mL, at least about 400 RU/mL, at least about 450 RU/mL, at least about 500 RU/mL, at least about 550 RU/mL, at least about 600 RU/mL, at least about 650 RU/mL, at least about 700 RU/mL, at least about 750 RU/mL, at least about 800 RU/mL, at least about 850 RU/mL, at least about 900 RU/mL, or at least about 1,000 RU/mL; and
the iFGF23 level of the subject increases by at least about 10 pg/mL, at least about 20 pg/mL, at least about 30 pg/mL, at least about 40 pg/mL, at least about 50 pg/mL, at least about 60 pg/mL, at least about 70 pg/mL, at least about 80 pg/mL, at least about 90 pg/mL, at least about 100 pg/mL, at least about 110 pg/mL, at least about 120 pg/mL, at least about 130 pg/mL, at least about 140 pg/mL, at least about 150 pg/mL, at least about 160 pg/mL, at least about 170 pg/mL, at least about 180 pg/mL, at least about 190 pg/mL, at least about 200 pg/mL, at least about 250 pg/mL, at least about 300 pg/mL, at least about 350 pg/mL, at least about 400 pg/mL, at least about 450 pg/mL, at least about 500 pg/mL, at least about 550 pg/mL, at least about 600 pg/mL, at least about 650 pg/mL, at least about 700 pg/mL, at least about 750 pg/mL, at least about 800 pg/mL, at least about 850 pg/mL, at least about 900 pg/mL, or at least about 1,000 pg/mL.

20. The method of claim 1, wherein
the iron carbohydrate complex comprises an iron dextran;
the phosphorus level of the subject decreases by at least about 0.001 mg/dL, at least about at least about 0.005 mg/dL, at least about 0.01 mg/dL, at least about 0.15 mg/dL, or at least about 0.2 mg/dL; and
the cFGF23 level of the subject decreases by at least about 100 RU/mL, at least about 150 RU/mL, at least about 200 RU/mL, at least about 250 RU/mL, at least about 300 RU/mL, at least about 350 RU/mL, at least about 400 RU/mL, at least about 450 RU/mL, at least about 500 RU/mL, at least about 550 RU/mL, at least about 600 RU/mL, at least about 650 RU/mL, at least about 700 RU/mL, at least about 750 RU/mL, at least about 800 RU/mL, at least about 850 RU/mL, at least about 900 RU/mL, or at least about 1,000 RU/mL.

21. The method of claim 1, wherein the subject is a mammalian subject.

22. The method of claim 1, wherein the subject is a human subject.

23. A method of modulating phosphorus or FGF23 in a subject comprising:
administering an iron carbohydrate complex to a subject in need thereof;
wherein,
a phosphorus level of the subject decreases;
a fractional excretion of phosphate of the subject increases;
a C-terminal FGF23 (cFGF23) level of the subject decreases; or
an intact FGF23 (iFGF23) level of the subject increases.
